(19) United States
(12) Patent Application Publication
(10) Pub. No.: US 2025/0041058 A1
(43) Pub. Date: Feb. 6, 2025

(54) SYSTEMS AND TECHNIQUES FOR PERCUTANEOUSLY CONTROLLING SLACK IN A TETHER

(71) Applicant: Edwards Lifesciences Innovation (Israel) Ltd., Caesarea (IL)

(72) Inventors: Gidon Pesach, Kfar Vitkin (IL); Yaron Herman, Givat Ada (IL)

(21) Appl. No.: 18/926,950

(22) Filed: Oct. 25, 2024

Related U.S. Application Data
(63) Continuation of application No. PCT/IB2023/054169, filed on Apr. 24, 2023.
(60) Provisional application No. 63/336,489, filed on Apr. 29, 2022.

(51) Int. Cl. A61F 2/24

(52) U.S. Cl. CPC A61F 2/2436

(57) ABSTRACT

Techniques for controlling slack in a tether of an implant during implantation of the implant are described. An implant comprises a spool, and a tether coupled to the spool. A delivery tool is configured to transluminally deliver the implant to the heart. The delivery tool can include a shaft, a handle, and a depth-stopper that extends axially from the handle. The shaft is coupled to the spool. The handle includes a controller and is coupled to a proximal part of the shaft such that distal movement of the handle can move the spool distally into the heart. The controller is operatively coupled to the spool and the depth-stopper such that operation of the controller adjusts an amount of winding of the tether around the spool and also adjusts axial extension of the depth-stopper from the handle. Other implementations are also described.

### CROSS-REFERENCE TO RELATED APPLICATIONS

The present application is a continuation of International Patent Application No. PCT/IB2023/054169, filed Apr. 24, 2023, which claims the benefit of U.S. Patent Application No. 63/336,489, filed Apr. 29, 2022, the entire disclosures all of which are incorporated by reference for all purposes.

### BACKGROUND

Dilation of the annulus of a native valve can prevent the valve leaflets from fully coapting when the valve is closed. Regurgitation of blood in the wrong direction through a valve (e.g., from a left ventricle into a left atrium, etc.) results in increased total stroke volume and decreased cardiac output, and ultimate weakening of the heart (e.g., a weakening of a ventricle and also a volume overload and a pressure overload of the left atrium).

Chronic or acute ventricular dilatation can lead to papillary muscle displacement with increased leaflet tethering due to tension on chordae tendineae, as well as annular dilatation.

Several techniques and implants for treating such conditions, as well as other conditions, utilize implantation of a tether or similar elongate and/or tensile member.

### SUMMARY

This summary is meant to provide some examples and is not intended to be limiting of the scope of the disclosure in any way. For example, any feature included in an example of this summary is not required by the claims, unless the claims explicitly recite the features. Also, the features described can be combined in a variety of ways. Various features and steps as described elsewhere in this disclosure can be included in the examples summarized here.

Methods and systems of controlling slack in a tether of an implant during implantation of the implant are disclosed. For example, the methods and systems described herein can relate to methods and systems for controlling slack in the tether during advancement of a component of the implant from a first site within the heart towards a second site within the heart.

In some implementations, the implant can be a tissue-adjusting implant such as a leaflet-augmenting implant, a prosthetic chorda implant, or an annuloplasty implant, and the tether can be used to draw together, contract, and/or restrict movement of tissue of the heart.

In some implementations, the tether can be used to tether a first component of the implant (which can be secured to tissue at a first site in the heart) to a second component of the implant (which can be secured to tissue at a second site in the heart), e.g., in order to draw together and/or restrict movement of the first component and/or the second component. Alternatively or additionally, the tether can be configured to contract a component of the implant.

In some implementations, the tether (e.g., a first end of the tether, which can be connected to a first component of the implant) is secured to the heart at a first site, and a second component of the implant, coupled to the tether (e.g., to a second end of the tether), is then advanced away from the first site towards a second site of the heart, e.g., in order to secure the tether at the second site (e.g., by securing the second component at the second site).

In some implementations, devices and techniques are described for reducing excess slack in the tether during implantation of the implant (e.g., during advancement of the second component towards the second site), e.g., in order to reduce a likelihood of tangling and/or ensnarement of the tether. Similarly, in some implementations, devices and techniques are described for providing sufficient slack in the tether during implantation of the implant (e.g., during advancement of the second component towards the second site), e.g., in order to reduce a likelihood of the tether tugging on, or becoming disconnected from, the first site. For example, some devices and techniques are described for ensuring that the tether is let out progressively during movement of the second component toward the second site.

In some implementations, the second component includes a winch that includes a spool around which a distal portion of the tether is wound.

In some implementations, a delivery tool (e.g., that can be used for delivery, positioning, and implantation of an implant) can include an extracorporeal handle, coupled to a flexible shaft (e.g., a catheter or sheath), which extends from the handle to the implant. Due to this coupling, the implant (e.g., the winch of the implant) can be moved within the vasculature (e.g., within the heart) by advancing and withdrawing the handle, and thereby the delivery tool. For example, pushing the handle distally pushes the winch distally through the vasculature, and pulling the handle proximally pulls the winch proximally within the vasculature.

In some implementations, the handle can be adapted to control the winch in order to control the amount of the tether that is contained by/let out from the winch. For example, the handle can include a winch controller, operatively coupled to the winch.

In some implementations, the handle is arranged such that axial movement of the handle is limited (e.g., by a limiter) in a manner that is linked to operation of the winch, in order to ensure that the tether has sufficient, but not excess, slack as the winch is moved through the vasculature by the axial movement of the handle.

In some implementations, after the tether has been secured to the first site, axial movement of the handle in a direction that moves the winch towards the second site may be limited. Letting out some of the tether from the winch modifies the limit in a manner that allows further advancement in this direction. In some implementations, this is achieved by the delivery tool including a limiter that defines limits for axial movement of the handle, and links these limits to the amount of the tether that has been let out from the winch.

In some implementations, this limiting is provided by the handle including a depth-stopper that extends axially from the handle, and that serves as the limiter (or a component thereof).

In some implementations, the handle can be limited from moving axially by the depth stopper abutting a mount or other component in the depth-stopper's path.

In some implementations, as well as the winch controller being operatively coupled to the spool, it is also operatively coupled to the depth-stopper, such that operation of the winch controller in a manner that operates the winch, also adjusts axial extension of the depth-stopper with respect to the handle—and thereby adjusts the limit to which the handle is axially movable (e.g., with respect to the mount).

Alternatively or additionally, the distal part of the shaft (e.g., the part of the shaft adapted to position the implant within the heart) can include a telescopic assembly (e.g., a distal portion and a proximal portion which are axially slidable with respect to each other).

In some implementations, the handle includes a shaft controller (e.g., a shaft extender), operatively coupled to the shaft such that operation of the shaft controller reversibly extends the distal portion of the shaft distally from the proximal portion of the shaft. Thus, in some implementations, advancement of the winch from the first site to the second site can be achieved by telescopically lengthening the shaft within the heart.

In some implementations, the shaft controller can be operatively coupled to the winch controller in a manner that limits telescopic lengthening according to an amount of tether let out from the winch-thereby defining a limiter having similar functionality to that described hereinabove, albeit without a depth stopper.

In some implementations, the operative coupling between the shaft controller and the winch controller can be such that operating the shaft controller to lengthen the shaft within the heart (e.g., such that the winch moves towards the second site) automatically operates the winch controller (e.g., such that more tether is exposed within the heart). Such coupling between the shaft controller and the winch controller may automatically ensure that the tether is let out at the rate required by the movement of the winch, thus preventing excess slack in the tether and/or tugging of the tether at the first site.

Additional techniques for controlling excess slack in a tether of an implant during implantation of the implant are also described. For example, and similarly to the methods and systems described hereinabove, the current techniques can be used for preventing excess slack in the tether during advancement of part (e.g., an end portion) of the tether, from a first site within the heart to which the tether is attached, towards a second site within the heart, e.g., in order to reduce a likelihood of tangling and/or ensnarement of the tether. Similarly, devices and techniques described can be used to reduce a likelihood of the tether tugging on, or becoming disconnected from, the first site.

In some implementations, during advancement of the implant from the first site towards the second site, a portion of the tether is confined within a channel (e.g., within a lumen of a catheter), and is released progressively as the end portion of the tether is advanced to the second site. For example, a middle portion of the tether can extend, as a bight into the channel and back out of the channel, and towards the end portion of the tether. In some implementations, confining this middle portion of the tether may prevent tangling and/or ensnarement of the tether within the heart during the advancement and/or implantation of the implant.

In some implementations, this confinement is achieved by a retaining member that is at least partially disposed within the channel. A portion (e.g., an end portion) of the retaining member can be constrained within the channel to define a hook by the inner wall of the channel, the hook being hooked to the bight, thereby retaining the middle portion within the channel.

In some implementations, the retaining member includes a deformable portion that is deformable such that pulling the tether (e.g., as a result of moving the end portion of the tether toward the second site) can pull the hook towards a distal opening of the channel, thereby increasing the amount of tether exposed within the heart. That is, the tether can be progressively released from the channel as it is pulled. Once a predetermined amount of pulling has occurred, the hook becomes exposed out of the opening of the channel, and responsively opens and unhooks from the bight (e.g., is pulled open by the tether).

In some implementations, the predetermined amount of pulling at which the hook becomes exposed may be dependent on the deformability (e.g., flexibility and/or stretchability) of the retaining member and/or the length of the channel.

In some implementations, the deformable portion of the retaining member is elastically deformable, such that if the tether is pulled, but not to the predetermined amount of pulling (and therefore the bight of the tether remains hooked by the hook within the channel), relaxing of the pulling results in the retaining member drawing the tether back into the channel. For example, the deformable portion can be a spring (e.g., an extension spring).

In some implementations, the predetermined amount of pulling at which the hook becomes exposed may be dependent on the spring constant of the spring and/or the length of the channel. Such an elastically-deformable portion may allow repeated repositioning of the end of the tether within the heart, while maintaining tension on the tether (e.g., a small amount of tension, sufficient to reduce slack without interfering with the repositioning).

In some implementations, the retaining member is plastically deformable. Thus, in some implementations, the predetermined amount of pulling at which the hook becomes exposed may be dependent on the plasticity of the retaining member and/or the length of the channel.

In some implementations, the retaining member comprises an elongate element (e.g., a thread, a suture, a line, or a wire), a portion of the elongate element being constrained by the channel to define the hook. In some implementations, the same elongate element also is shaped to define the deformable portion (e.g., a spring) of the retaining member, e.g., a unitary elongate member (e.g., a single piece of wire) defines both the deformable portion and the hook.

In some implementations, rather than an elongate element that is constrained by the channel to define a hook, a retaining member is provided comprising an elongate member (e.g., a thread, a suture, a line, or a wire) that extends distally through the channel, loops around the bight of the tether, and returns proximally through the channel.

In some implementations, one or both ends of the elongate member are disposed outside of a proximal opening of the channel, and can be disposed at a proximal portion of the system that is configured to remain outside of the subject.

In some implementations, pulling the tether (e.g., the end portion of the tether) within the heart pulls the loop towards the distal opening of the channel, increasing the amount of tether exposed from the channel within the heart.

Once the advancement of the end portion is determined to be complete (e.g., once the end portion arrives at the second site), the bight can be released from the elongate member by releasing the loop from around the bight (e.g., by releasing one end of the elongate member and pulling the other end of the elongate member).

In some implementations, an end of the elongate member is coupled to a deformable member, e.g., in order to maintain tension on the tether (e.g., a small amount of tension, sufficient to reduce slack).

The above method(s)/procedure(s) and steps herein can be performed on a living animal or on a simulation, such as on a cadaver, cadaver heart, simulator (e.g., with the body parts, heart, tissue, etc. being simulated), etc.

In accordance with some implementations, a system (e.g., a system for use with or usable with a heart of a subject), the system including an implant and a delivery tool. In some implementations, the implant includes (i) a winch, including a spool, and (ii) a tether, coupled to the spool. In some implementations, the delivery tool is configured to transluminally deliver the implant to the heart.

In some implementations, the delivery tool comprises a shaft, coupled to the winch. The shaft can be configured to transluminally position the winch within the heart.

In some implementations, the delivery tool comprises a handle, including a winch controller, which can be coupled to a proximal part of the shaft such that distal movement of the handle moves the winch distally into the heart.

In some implementations, the proximal part of the shaft defines an axis of the delivery tool.

In some implementations, the delivery tool includes a depth-stopper, which can extend outwardly (e.g., axially) from the handle.

In some implementations, the winch controller is operatively coupled to the winch and to the depth-stopper such that operation of the winch controller in a manner that adjusts an amount of winding of the tether around the spool, also adjusts axial extension of the depth-stopper from the handle.

In some implementations, the depth-stopper includes a rod.

In some implementations, the depth-stopper extends parallel to the axis.

In some implementations, the shaft is configured to transluminally position the winch within the heart while extending, from the handle, through the vasculature of the subject, to the winch within the heart.

In some implementations, the winch controller includes a control knob, and is operatively coupled to the winch and to the depth-stopper such that rotation of the control knob in a manner that adjusts an amount of winding of the tether around the spool, also adjusts axial extension of the depth-stopper from the handle.

In some implementations, the winch controller is operatively coupled to the winch and to the depth-stopper via a rotary-to-linear mechanism.

In some implementations, the rotary-to-linear mechanism includes a worm drive.

In some implementations, the implant includes an anchor, coupled to the winch, and configured to anchor the winch to tissue at a site in the heart.

In some implementations, the anchor is a second anchor, the site is a second site, and the implant further includes a first anchor, coupled to a first end portion of the tether, and configured to anchor the first end portion of the tether to tissue at a first site within the heart. In some implementations, the shaft is configured to transluminally advance the winch and the second anchor towards the second site while the first anchor remains anchored at the first site.

In some implementations, the first site is a leaflet site at a leaflet of a valve of the heart and the first anchor is a leaflet anchor adapted to be anchored to the leaflet. In some implementations, the second site is a ventricular site in a ventricle downstream of the valve, the second anchor is a ventricular anchor, and the shaft is configured to transluminally advance the winch and the ventricular anchor towards the ventricular site while the first anchor remains anchored at the leaflet site.

In some implementations, the delivery tool has a state in which the depth-stopper extends distally from the handle.

In some implementations, the winch controller is operatively coupled to the winch and to the depth-stopper such that operation of the winch controller in a manner that decreases the amount of winding of the tether around the spool, also moves the depth-stopper proximally with respect to the handle.

In some implementations, the winch controller is operatively coupled to the winch and to the depth-stopper such that operation of the winch controller in a manner that increases the amount of winding of the tether around the spool, also moves the depth-stopper distally with respect to the handle.

In some implementations, the winch controller is operatively coupled to the winch and to the depth-stopper such that operation of the winch controller in a manner that decreases the amount of winding of the tether around the spool, also moves the depth-stopper distally with respect to the handle.

In some implementations, the winch controller is operatively coupled to the winch and to the depth-stopper such that operation of the winch controller in a manner that increases the amount of winding of the tether around the spool, also moves the depth-stopper proximally with respect to the handle.

In some implementations, the state is a first state, and the delivery tool has a second state in which the depth-stopper extends proximally from the handle.

In some implementations, in the state, the depth-stopper extends from the handle simultaneously distally and proximally.

In some implementations, the system further includes a mount in which the handle is mountable, and which, while the handle is mounted in the mount, is configured to obstruct axial movement of the depth-stopper along an axial path by being disposed in the axial path.

In some implementations, the mount extends perpendicular to the axis.

In some implementations, the winch controller is operatively coupled to the winch and to the depth-stopper such that (i) operation of the winch controller in a manner that decreases an amount of winding of the tether around the spool, also moves the depth-stopper proximally with respect to the mount, and (ii) operation of the winch controller in a manner that increases an amount of winding of the tether around the spool, also moves the depth-stopper distally with respect to the mount.

In some implementations, the winch controller is operatively coupled to the winch and to the depth-stopper such that: (i) operation of the winch controller in a manner that increases an amount of winding of the tether around the spool, also moves the depth-stopper proximally with respect to the mount, and (ii) operation of the winch controller in a manner that decreases an amount of winding of the tether around the spool, also moves the depth-stopper distally with respect to the mount.

In some implementations, the winch controller is operatively coupled to the winch and to the depth-stopper such that operation of the winch controller in the manner that adjusts the amount of winding of the tether around the spool, also adjusts an axial position of the depth-stopper with respect to the mount.

In some implementations, the mount includes: (i) a distal stop that, while the handle is mounted in the mount, is positioned to obstruct movement of the depth-stopper distally by being disposed in the axial path; (ii) a proximal stop that, while the handle is mounted in the mount, is positioned to obstruct movement of the depth-stopper proximally by being disposed in the axial path.

In some implementations, the winch controller is operatively coupled to the winch and to the depth-stopper such that: (i) operation of the winch controller in a manner that decreases the amount of winding of the tether around the spool, also moves the depth-stopper axially away from the distal stop, and (ii) operation of the winch controller in a manner that increases the amount of winding of the tether around the spool, also moves the depth-stopper axially toward the distal stop.

In some implementations, the winch controller is operatively coupled to the winch and to the depth-stopper such that: (i) operation of the winch controller in a manner that decreases the amount of winding of the tether around the spool, also moves the depth-stopper axially toward the distal stop, and (ii) operation of the winch controller in a manner that increases the amount of winding of the tether around the spool, also moves the depth-stopper axially away from the distal stop.

In accordance with some implementations, a method (e.g., for use with or usable with a heart of a subject), the method including using a delivery tool having a handle, transluminally advancing, to the heart of the subject, a winch operatively coupled to a tether.

In some implementations, the method includes attaching the tether to tissue at a first site within the heart.

In some implementations, the method includes (i) operating a winch controller of the handle to actuate the winch and move, in a first axial direction with respect to the handle, a depth-stopper that extends axially from the handle; and/or (ii) while the tether remains coupled to the tissue at the first site, advancing the winch away from the first site by advancing the handle of the delivery tool in a second axial direction that is opposite to the first direction.

In some implementations, advancing the winch away from the first site includes advancing the winch away from the first site subsequently to operating a winch controller of the handle to actuate the winch.

In some implementations, the method further includes mounting the handle in a mount.

In some implementations, mounting the handle in the mount includes mounting the handle in the mount subsequently to the step of transluminally advancing the winch to the heart, and prior to the step of operating the winch controller.

In some implementations, operating the winch controller includes operating the winch controller to move the depth-stopper in the first axial direction with respect to the handle such that the depth-stopper approaches a stop defined by the mount.

In some implementations, operating the winch controller includes operating the winch controller until the depth-stopper abuts the stop.

In some implementations, advancing the handle of the delivery tool in the second axial direction includes advancing the handle of the delivery tool in the second axial direction such that the depth-stopper approaches a stop defined by the mount.

In some implementations, advancing the handle of the delivery tool in the second axial direction includes advancing the handle of the delivery tool in the second axial direction until the depth-stopper abuts the stop.

In some implementations, the steps of operating the winch controller and advancing the winch are performed iteratively at least until the winch reaches a second site within the heart.

In some implementations, the method further includes anchoring the winch to tissue at the second site.

In some implementations, the method further includes, subsequently to anchoring the winch to the tissue at the second site, adjusting an amount of the tether that is let out from the winch by operating the winch controller to actuate the winch.

In some implementations, the method further includes, subsequently to anchoring the winch to the tissue at the second site, adjusting tension on the tether by operating the winch controller to actuate the winch.

The above methods can be performed on a living animal or on a simulation, such as on a cadaver, cadaver heart, simulator (e.g., with the body parts, tissue, etc. being simulated), etc.

In accordance with some implementations, a system (e.g., for use with or usable with a heart of a subject), the system including: (i) an implant, including a spool and a tether coupled to the spool; and (ii) a delivery tool, configured to transluminally deliver the implant to the heart.

In some implementations, the delivery tool includes a shaft, coupled to the spool, and configured to transluminally position the spool within the heart.

In some implementations, the delivery tool includes (i) a mount, and/or (ii) a handle, mountable on the mount, and coupled to a proximal end of the shaft such that axial movement of the handle with respect to the mount axially moves the shaft with respect to the mount.

In some implementations, the delivery tool includes a limiter, configured to define a limit by which the handle is axially movable with respect to the mount.

In some implementations, the handle includes a controller that operatively couples the spool to the limiter such that operation of the controller in a manner that adjusts an amount of winding of the tether around the spool, also modifies the limit.

In some implementations, the limiter comprises a depth-stopper.

In some implementations, the delivery tool comprises a shaft controller that is adapted to lengthen the shaft within the heart.

In some implementations, operating the shaft controller to lengthen the shaft within the heart automatically operates the controller to increase the amount of tether exposed within the heart.

In some implementations, the shaft is configured to transluminally position the spool within the heart while extending, from the handle, through vasculature of the subject, to the spool within the heart.

In some implementations, the controller comprises a control knob, and is operatively coupled to the spool and to the limiter such that rotation of the control knob adjusts the amount of winding of the tether around the spool, and also modifies the limit.

In some implementations, the controller is operatively coupled to the spool and to the limiter via a rotary-to-linear mechanism.

In some implementations, the rotary-to-linear mechanism comprises a worm drive.

In some implementations, the implant comprises an anchor, coupled to the spool, and/or configured to anchor the spool to tissue at a site in the heart.

In some implementations, the anchor is a second anchor, and the site is a second site. In some implementations, the implant further comprises a first anchor, coupled to a first end portion of the tether, and configured to anchor the first end portion of the tether to tissue at a first site within the heart. In some implementations, the shaft is configured to transluminally advance the spool and the second anchor towards the second site while the first anchor is anchored at the first site.

In some implementations, the first site is a leaflet site at a leaflet of a valve of the heart, the first anchor is adapted to be anchored to the leaflet site, the second site is a ventricular site in a ventricle downstream of the valve, and/or the second anchor is adapted to be anchored to the ventricular site.

In some implementations, the shaft is configured to transluminally advance the spool and the second anchor towards the ventricular site while the first anchor is anchored at the leaflet site.

In some implementations, while the handle is mounted on the mount, the mount is disposed in the axial path such that the mount obstructs the axial movement of the handle upon the handle reaching the limit.

In some implementations, the mount comprises a distal stop that, while the handle is mounted on the mount, is positioned to obstruct movement of the limiter distally by being disposed in the axial path.

In some implementations, a proximal stop that, while the handle is mounted on the mount, is positioned to obstruct movement of the limiter proximally by being disposed in the axial path.

In some implementations, the handle is mountable on the mount subsequently to the shaft transluminally positioning the spool within the heart.

In accordance with some implementations, a system for controlling slack in a tether during implantation of the tether in a heart of a subject (e.g., of a living subject or simulation), the system including: (i) the tether, the tether having a first end portion, a second end portion, and defining a bight therebetween (e.g., shapable to form an open loop therebetween); and (ii) a delivery tool, configured to secure the first end portion of the tether and the second end portion of the tether within the heart.

In some implementations, the delivery tool includes a channel having an inner wall and a distal opening.

In some implementations, the delivery tool includes a retaining member, constrained by the inner wall to define a hook within the channel.

In some implementations, the bight of the tether is confined within the channel by being hooked onto the hook.

In some implementations, the retaining member is deformable responsively to pulling of the tether out of the distal opening in a manner that moves the hook toward the distal opening.

In some implementations, the retaining member is configured such that, upon the hook exiting the channel via the distal opening, the hook responsively opens and unhooks from the bight.

In some implementations, the retaining member includes a deformable portion that is plastically deformable responsively to the pulling of the tether.

In some implementations: (i) the delivery tool includes a shaft, and an extracorporeal handle coupled to a proximal part of the shaft, and/or (ii) a distal part of the shaft is coupled to the tether such that, while the shaft is disposed through the vasculature of the subject to the tether within the heart, the shaft is configured to transluminally position the tether within the heart by pulling the tether out of the distal opening in the manner that moves the hook toward the distal opening.

In some implementations the delivery tool includes a tube defining the channel, and the shaft extends through the tube, and is configured to transluminally position the tether within the heart by the shaft sliding distally outwards from the tube in a manner that pulls the tether distally out of the distal opening.

In some implementations, the handle is coupled to a proximal part of the tube, and the handle is configured to slide the shaft distally outward from the tube.

In some implementations, the retaining member includes a deformable portion that is elastically deformable responsively to the pulling of the tether, and is adapted to draw the tether back into the channel, responsively to relaxing of the pulling of the tether.

In some implementations, the deformable portion includes a spring.

In some implementations, the retaining member includes a unitary elongate member that is shaped to define the deformable portion and the hook.

In some implementations, the system includes an implant that includes the tether, a first component, and a second component, the tether tethering the first component to the second component.

In some implementations, the delivery tool is configured to: (A) anchor the first component at a first site within the heart, and (B) subsequently, advance the second component from the first site toward a second site within the heart while (i) the first component remains anchored at the first site, and (ii) the bight of the tether remains confined within the channel by the hook of the retaining member.

In some implementations, the second component includes a winch, operatively coupled to the tether.

In some implementations, the first site is a leaflet site at a leaflet of a valve of the heart, and the first component is a leaflet anchor adapted to be anchored to the leaflet. In some implementations, the second site is a ventricular site in a ventricle downstream of the valve, and the second component is a ventricular anchor adapted to be anchored to ventricular tissue. In some implementations, the delivery tool is configured to transluminally advance the ventricular anchor towards the ventricular site while the first component remains anchored at the leaflet site.

In accordance with some implementations, a method (e.g., for use with or usable with a heart of a subject), the method including: (i) using a delivery tool that defines a channel at a distal portion thereof, advancing a tether to the heart while a bight of the tether is retained within the channel by a hook disposed in the channel; and (ii) while the distal portion of the delivery tool is disposed within the heart, progressively pulling the tether out of an opening of the channel such that the hook moves towards the opening of the channel.

In some implementations, the method includes subsequently, releasing the bight from the hook by pulling the tether sufficiently to pull the hook out of the opening such that the hook responsively unhooks from the bight.

In some implementations, part of the retaining member is fixed with respect to the channel at an attachment site, and progressively pulling the tether out of the opening of the channel includes progressively pulling the tether away from the attachment site and out of the opening of the channel.

In some implementations: (i) the hook is a component of a retaining member that further includes a deformable portion coupled to the hook, and (ii) progressively pulling the tether out of the opening of the channel includes progressively pulling the tether out of the opening of the channel such that the deformable portion deforms as the hook moves towards the distal opening.

In some implementations, pulling the tether sufficiently to pull the hook out of the opening includes withdrawing the delivery tool proximally away from the tether while the tether remains within the heart.

In some implementations: the tether is a component of an implant, the implant further includes an anchor coupled to the tether, and/or the method further includes anchoring the anchor to tissue of the heart.

In some implementations, anchoring the anchor to tissue of the heart includes anchoring the anchor to tissue of the heart subsequently to unhooking the hook from the bight.

In some implementations, anchoring the anchor to tissue of the heart includes anchoring the anchor to tissue of the heart prior to unhooking the hook from the bight.

In some implementations, the implant further includes a winch, and anchoring the anchor includes anchoring the winch to tissue by anchoring the anchor.

In some implementations, the method further includes, subsequently to anchoring the winch to the tissue, adjusting an amount of the tether that is let out from the winch by actuating the winch.

In some implementations, the method further includes, subsequently to anchoring the winch to the tissue, adjusting tension on the tether by actuating the winch.

In some implementations: (i) the anchor is a second anchor, the implant further including a first anchor coupled to the tether, and (ii) anchoring the anchor includes anchoring the second anchor at a second site within the heart, such that the tether extends from the second anchor, anchored at the second site, to the first anchor.

In some implementations, the method further includes anchoring the first anchor at a first site within the heart, prior to anchoring the second anchor at the second site.

In some implementations, progressively pulling the tether out of the opening of the channel includes progressively pulling the tether out of the opening of the channel by advancing the second anchor away from the first site toward the second site, subsequently to anchoring the first anchor.

The above methods can be performed on a living animal or on a simulation, such as on a cadaver, cadaver heart, simulator (e.g., with the body parts, tissue, etc. being simulated), etc.

In accordance with some implementations, a system for controlling slack in a tether of an implant during implantation of the tether in a heart of a subject (e.g., of a living subject or simulation), the system including: (i) the implant, including the tether, the tether having a first end portion, a second end portion, and a bight therebetween; and (ii) a delivery tool, configured to secure the first end portion of the tether and the second end portion of the tether within the heart.

In some implementations, the delivery tool includes an extracorporeal control portion.

In some implementations, the delivery tool includes a shaft extending, from the extracorporeal control portion to the implant, and adapted to position the implant within the heart.

In some implementations, the delivery tool includes, at a distal part of the shaft, a channel having a distal opening; and an elongate member, coupled to the extracorporeal control portion, and extending away from the extracorporeal control portion to form a loop within the channel.

In some implementations, the system has a delivery state in which: (i) the bight of the tether is disposed within the channel, (ii) the first and second end portions of the tether are disposed outside of the distal opening of the channel, and (iii) the elongate member confines the bight of the tether within the channel by being looped onto the bight.

In some implementations, at least a part of the elongate member is decouplable from the extracorporeal control portion to facilitate release of the bight from the elongate member via unlooping the elongate member from the bight.

In some implementations, in the delivery state, both a first end and a second end of the elongate member are coupled to the extracorporeal control portion.

In some implementations, the implant further includes a first component and a second component, the tether tethering the first component to the second component.

In some implementations, the delivery tool is configured to: (A) anchor the first component at a first site within the heart, and (B) subsequently, advance the second component away from the first site toward a second site within the heart while (i) the first component remains anchored at the first site, and (ii) the bight of the tether remains confined within the channel by the elongate member being looped onto the bight.

In some implementations, the second component includes a winch, operatively coupled to the tether.

In some implementations, (i) the first site is at a leaflet of a valve of the heart, and the first component is a leaflet anchor adapted to be anchored to the leaflet. In some implementations, the second site is in a ventricle downstream of the valve, the second component is a ventricular anchor adapted to be anchored to tissue of the ventricle, and the delivery tool is configured to transluminally advance the ventricular anchor towards the second site in the ventricle while the first component remains anchored to the leaflet.

In some implementations, the delivery tool includes a tube, the channel is fixed with respect to the tube, and the shaft extends through the tube, and is configured to transluminally position the tether within the heart by the shaft sliding distally through the tube in a manner that pulls the tether distally out of the distal opening of the channel.

In some implementations, the tube is shaped to define the channel.

In some implementations, the extracorporeal control portion is coupled to a proximal part of the tube, and is configured to slide the shaft distally through the tube.

In some implementations, the elongate member is reversibly coupled to a deformable member that deforms in response to pulling of the tether toward the distal opening.

In some implementations, the deformable member is at the extracorporeal control portion.

In some implementations, the deformable member is elastically deformable in a manner that configures the deformable member to draw the bight back into the channel responsively to relaxing of the pulling of the tether.

In some implementations, the deformable member is plastically deformable responsively to the pulling of the tether.

In some implementations, the deformable member includes a spring that deforms in response to pulling of the tether toward the distal opening.

In some implementations, the deformable member includes a torsion spring.

In some implementations, the extracorporeal control portion includes a bobbin, operatively coupled to the elongate member in a manner that configures the bobbin to unloop the elongate member from the bight by winding the elongate member around the bobbin.

In some implementations, the deformable member includes a torsion spring.

In some implementations, the torsion spring is disposed within the bobbin.

In some implementations, the torsion spring is disposed around the bobbin.

In some implementations, the part of the elongate member is coupled to the extracorporeal control portion in a manner that exposes the part of the elongate member, such that the part of the elongate member is decouplable from the extracorporeal control portion by cutting the part of the elongate member.

In some implementations, the part of the elongate member is decouplable from the extracorporeal control portion by cutting the part of the elongate member thereby producing a cut end of the elongate member. In some implementations, the part of the elongate member is decouplable from the extracorporeal control portion to facilitate the release of the bight from the elongate member via drawing the cut end distally through the channel such that the elongate member unloops from the bight.

In accordance with some implementations, a method (e.g., for use with or usable with a heart of a subject), the method including: advancing a tether to the heart using a delivery tool that defines a channel at a distal portion thereof, and while the distal portion of the delivery tool is disposed within the heart, progressively pulling a tether out of an opening of the channel such that the elongate member moves towards the opening of the channel.

In some implementations, advancing the tether to the heart using the delivery tool is done while: (i) a bight of the tether is retained within the channel by an elongate member looped onto the bight within the channel, and (ii) at least part of the elongate member is coupled to an extracorporeal control portion of the delivery tool.

In some implementations, the method includes subsequently, releasing the tether from the channel by: (i) decoupling at least a part of the elongate member from the extracorporeal control portion, and (ii) drawing the part of the elongate member distally through the channel such that the elongate member unloops from the bight.

In some implementations, decoupling the part of the elongate member from the extracorporeal control portion includes cutting the part of the elongate member.

In some implementations, advancing the tether to the heart includes advancing the tether to the heart while both a first end and a second end of the elongate member are coupled to the extracorporeal control portion.

In some implementations, the extracorporeal control portion includes a bobbin, operatively coupled to the first end of the elongate member, and drawing the part of the elongate member distally through the channel includes drawing the second end of the elongate member distally through the channel by winding the elongate member around the bobbin.

In some implementations, the part of the elongate member is the second end of the elongate member, and decoupling the part of the elongate member from the extracorporeal control portion includes cutting the second end of the elongate member.

In some implementations, the elongate member is a component of a retaining member that further includes a deformable member coupled to the elongate member, and progressively pulling the tether out of the opening of the channel includes progressively pulling the tether out of the opening of the channel such that the deformable member deforms as the elongate member moves towards the opening.

In some implementations, the deformable member is elastically deformable in a manner that configures the deformable member to draw the bight back into the channel responsively to relaxing of the pulling of the tether.

In some implementations, the deformable member includes a spring that deforms in response to pulling of the tether toward the distal opening, and progressively pulling the tether out of the opening of the channel includes progressively pulling the tether out of the opening of the channel such that the spring deforms as the elongate member moves towards the opening.

In some implementations, the extracorporeal control portion includes a bobbin, operatively coupled to the elongate member, and drawing the part of the elongate member distally through the channel includes winding the elongate member around the bobbin such that the part of the elongate member is drawn distally into the channel.

In some implementations, the tether is a component of an implant, the implant further includes an anchor coupled to the tether, and the method further includes using the delivery tool to anchor the anchor to tissue of the heart.

In some implementations, anchoring the anchor to tissue of the heart includes anchoring the anchor to tissue of the heart subsequently to releasing the tether from the channel.

In some implementations, anchoring the anchor to tissue of the heart includes anchoring the anchor to tissue of the heart prior to releasing the tether from the channel.

In some implementations, the implant further includes a winch, and anchoring the anchor includes anchoring the winch to the tissue by anchoring the anchor to the tissue.

In some implementations, the method further includes, subsequently to anchoring the winch to the tissue, adjusting an amount of the tether that is let out from the winch by actuating the winch.

In some implementations, the method further includes, subsequently to anchoring the winch to the tissue, adjusting tension on the tether by actuating the winch.

In some implementations, the anchor is a second anchor, the implant further including a first anchor coupled to the tether, and anchoring the anchor includes anchoring the second anchor at a second site within the heart, such that the tether extends from the second anchor, anchored at the second site, to the first anchor.

In some implementations, the method further includes anchoring the first anchor at a first site within the heart, prior to anchoring the second anchor at the second site.

In some implementations, progressively pulling the tether out of the opening of the channel includes progressively pulling the tether out of the opening of the channel by advancing the second anchor away from the first site toward the second site, subsequently to anchoring the first anchor.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the above methods can comprise (or additional methods comprise or consist of) sterilization of one or more systems, devices, apparatuses, components, etc. herein (e.g., with heat, radiation, ethylene oxide, hydrogen peroxide, etc.).

The features, components, steps, concepts, etc. described in examples in this summary and elsewhere in this disclosure can be combined in a variety of ways. Various features and steps as described elsewhere in this disclosure can be included in the examples summarized here.

### BRIEF DESCRIPTION OF THE DRAWINGS

FIGS. 1, 2A-B, and 3A-B are schematic illustrations of systems and techniques for controlling excess slack in a tether of an implant during implantation of the implant; and

FIGS. 4A-E and 5A-C are schematic illustrations of systems and techniques for controlling excess slack in a tether of an implant during implantation of the implant.

### DETAILED DESCRIPTION

Methods and systems for controlling slack in a tether of an implant during implantation of the implant are described. For example, the methods and systems described herein may relate to methods and systems for controlling slack in the tether during advancement of a component of the implant from a first site within the heart towards a second site within the heart.

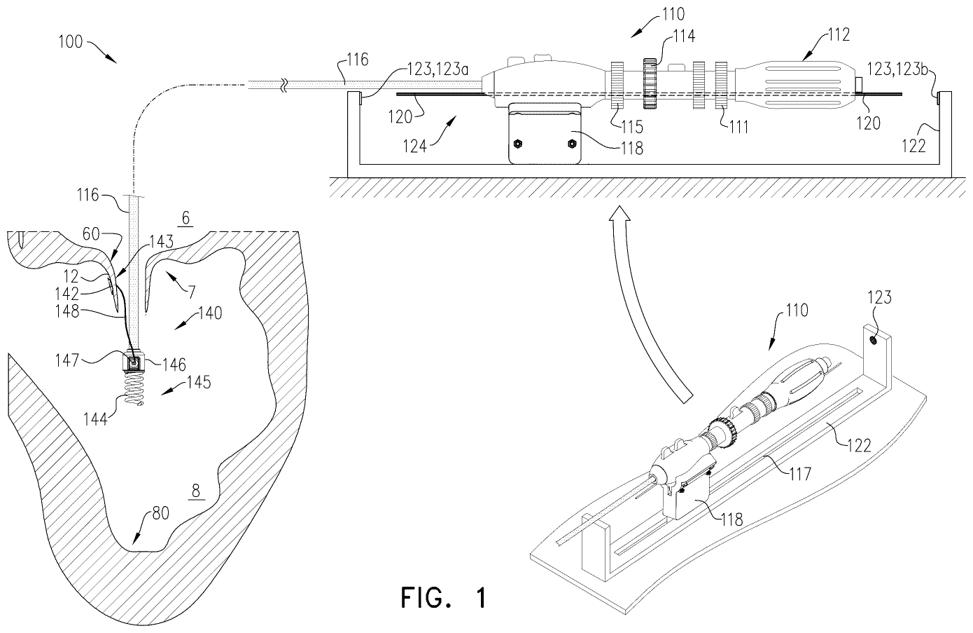

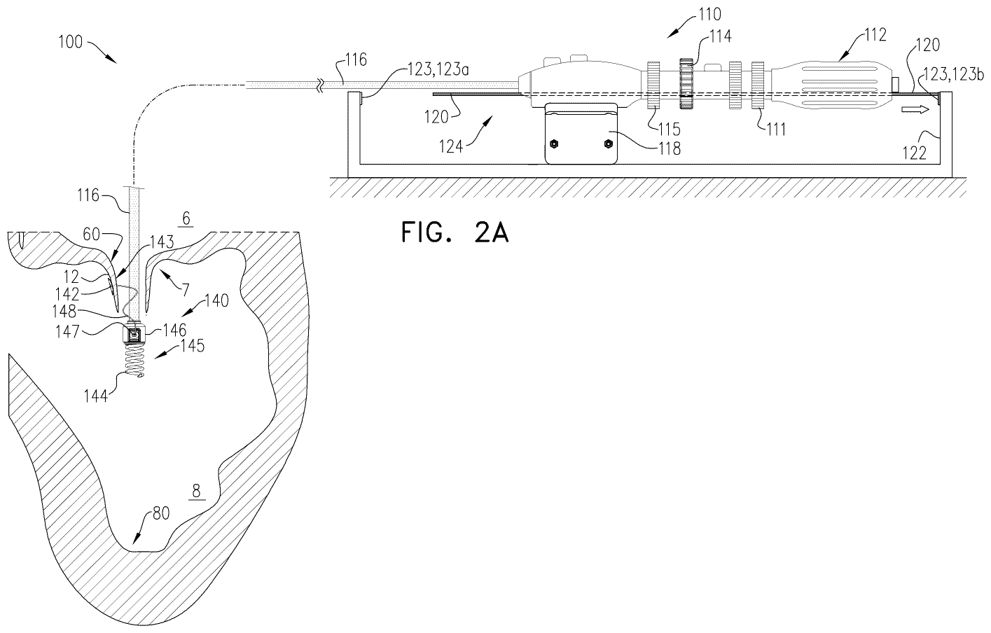

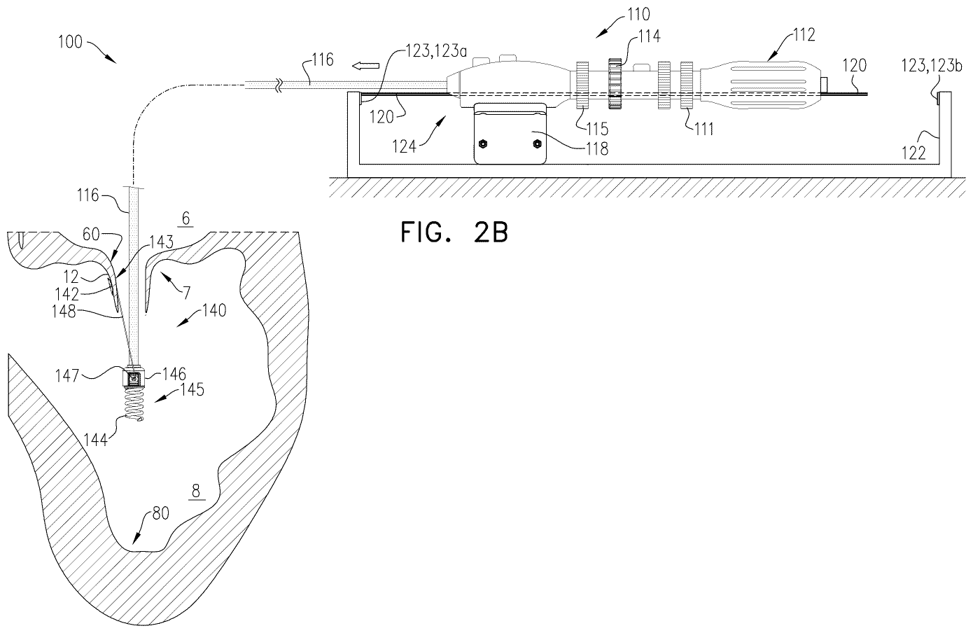

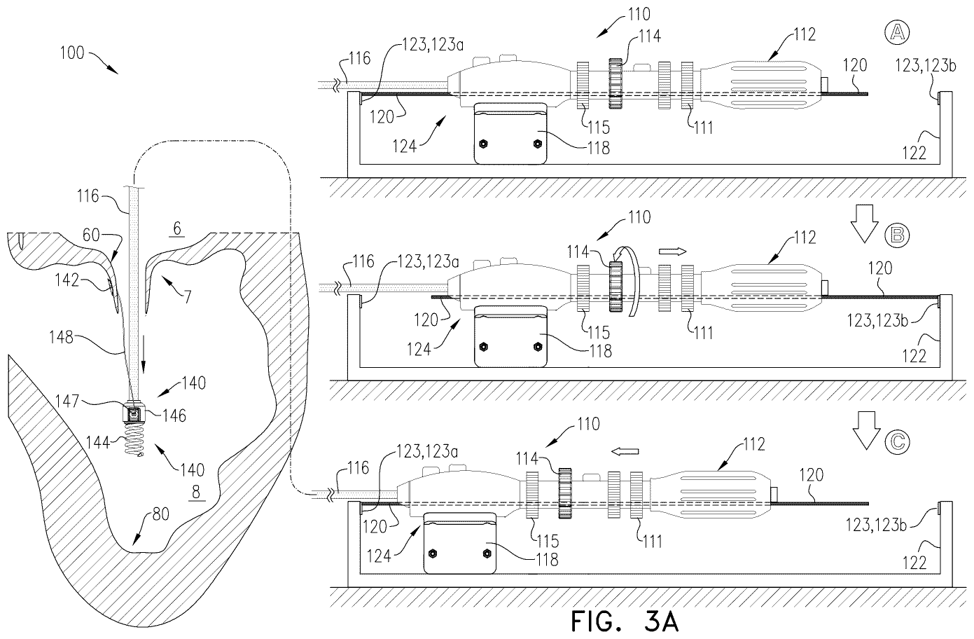

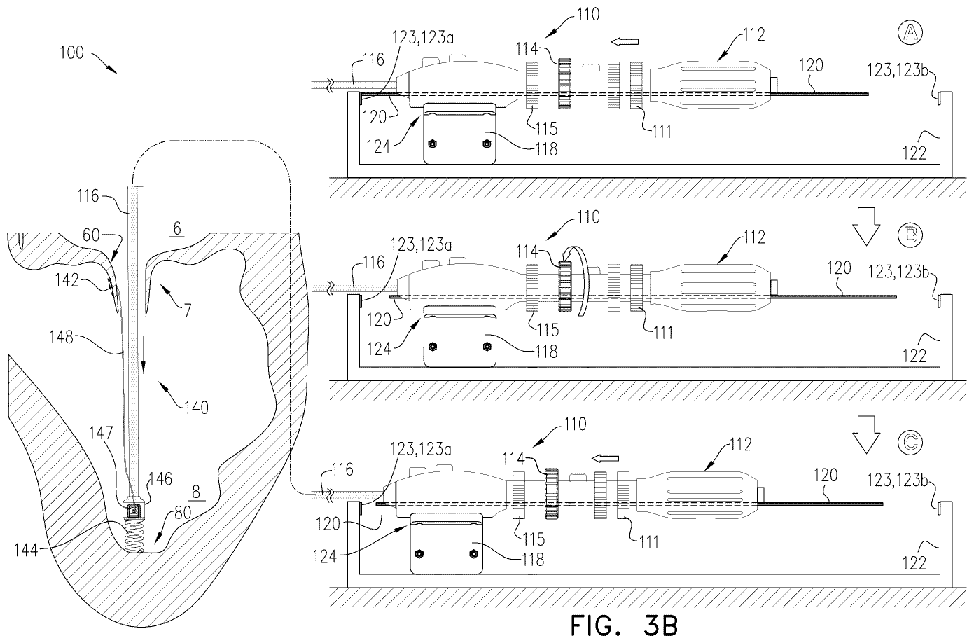

Reference is made to FIGS. 1, 2A-B, 3A-B, 4A-E, and 5A-C. In some implementations, a tether (e.g., a first end of the tether, which can be connected to a first component of the implant) is secured to the heart at a first site, and a second component of the implant, coupled to the tether (e.g., to a second end of the tether), is then advanced away from the first site towards a second site of the heart, e.g., in order to secure the tether at the second site (e.g., by securing the second component at the second site). Devices and techniques are described for reducing excess slack in the tether during implantation of the implant (e.g., during advancement of the second component towards the second site), e.g., in order to reduce a likelihood of tangling and/or ensnarement of the tether. Similarly, devices and techniques are described for providing sufficient slack in the tether during implantation of the implant (e.g., during advancement of the second component towards the second site), e.g., in order to reduce a likelihood of the tether tugging on, or becoming disconnected from, the first site. For example, devices and techniques are described for ensuring that the tether is let out progressively during movement of the second component toward the second site.

In some implementations, the implant can be a tissue-adjusting implant such as a leaflet-augmenting implant, a prosthetic chorda implant (as shown), and/or an annuloplasty implant, and the tether can be used to draw together, contract, and/or restrict movement of tissue of the heart. In some implementations, the tether can be used to tether a first component of the implant (which can be secured to tissue at a first site in the heart) to a second component of the implant (which can be secured to tissue at a second site in the heart), e.g., in order to draw together and/or restrict movement of the first component and/or the second component. Alternatively or additionally, the tether can be configured to contract a component of the implant.

The tether can be or comprise a line, wire, ribbon, rope, braid, contraction member, and/or suture. The tether can comprise (e.g., can be formed from) a metal, a polymer, and/or a natural fiber.

FIGS. 1, 2A-B, and 3A-3B are schematic illustrations of a system 100 comprising an implant 140, and a delivery tool 110 for use with the implant, in accordance with some implementations. Implant 140 comprises a winch 146, and a tether 148 coupled to the winch. For example, winch 146 can comprise a spool 147 around which tether 148 (e.g., one end of the tether) is wound. Winch 146 is configured to adjust an amount of tether 148 within the winch (e.g., wound around the spool) and, therefore, an amount of the tether disposed outside of the winch.

When system 100 is used, a part of tether 148 (e.g., the other end of the tether) is secured (e.g., anchored) to a first site of the tissue (such as a leaflet of a valve 7 of heart of a subject), and extends from that site to winch 146. System 100 is configured to move winch 146 within the heart with respect to the first site (e.g., away from the first site) and/or toward a second site (e.g., within a ventricle 8 of the heart), while providing sufficient but not excess slack on tether 148.

In the particular example shown, implant 140 is illustrated as an implant for reducing (e.g., eliminating) regurgitation through an atrioventricular valve 7 of the heart caused by suboptimal coaptation of the leaflets of the valve. Implant 140 can comprise a leaflet anchor 142, adapted to be attached to (e.g., anchored to) a leaflet 12 of valve 7 at a first site 60 of the heart. Leaflet anchor 142 can be considered to be (or to be a component of) an upstream assembly 143 of implant 140. Implant 140 further comprises a downstream assembly 145, comprising winch 146. In some implementations, and as shown, downstream assembly further comprises a ventricular anchor (e.g., an anchor) 144, adapted to be attached to the ventricle 8 downstream of valve 7 at a second site 80 of the heart subsequently to leaflet anchor 142 being attached to leaflet 12 at first site 60. A tether 148 tethers the downstream assembly to the upstream assembly.

Although implant 140 is described as comprising a leaflet anchor 142 and a ventricular anchor 144, it is to be understood that the techniques described hereinbelow could be used with other implants in which a winch, attached to a tether (e.g., one end of the tether), is moved with respect to a site at which the tether (e.g., the other end of the tether) is secured. Nonlimiting examples of such implants include chord repair implants, chord replacement implants, and annuloplasty implants.

Delivery tool 110 is provided and configured for delivery, positioning, and implantation of implant 140. Tool 110 comprises an extracorporeal handle 112 (e.g., an extracorporeal control portion of the delivery tool), coupled to a flexible shaft 116 (e.g., a catheter or sheath), which extends, from the handle, to implant 140. For example, a proximal part of shaft 116 can be coupled to the handle. During delivery and/or implantation of implant 140, the implant (e.g., downstream assembly 145, such as winch 146) can be advanced through, advanced out of, and/or coupled to shaft 116.

FIG. 1 shows leaflet anchor 142 (and thereby a first end of tether 148) anchored to a first site 60 (which is on a leaflet 12), and winch 146, within ventricle 8, coupled to a distal end of shaft 116, with a given amount of slack on tether 148.

The coupling of handle 112 to shaft 116 is such that implant 140 (e.g., winch 146 of the implant) can be moved within the vasculature (e.g., within the heart) by moving handle 112 axially, thereby moving the shaft and winch 146 therewith.

FIG. 1 shows system 100 (e.g., delivery tool 110 thereof) comprising a mount 122 that defines a track 117 within which a slider 118 is slidably mounted (e.g., to be axially slidable), and shows handle 112 being mountable on slider 118, e.g., such that axial movement of the handle slides the slider along the track. For example, once leaflet anchor 142 has been attached to the leaflet, and prior to ventricular anchor 144 being attached to the ventricle, the operator can mount handle 112 on slider 118, such that pushing the handle (and therefore the slider) distally along the track pushes winch 146 distally through the vasculature (e.g., downstream/deeper into ventricle 8; FIG. 2B), and pulling the handle and slider proximally along the track pulls the winch proximally within the vasculature (e.g., upstream/toward atrium 6; FIG. 2A).

In the example shown, pulling handle 112 along track 117 (and thereby winch 146) proximally from the position shown in FIG. 1 moves the winch toward first site 60, therefore increasing slack on tether 148 (FIG. 2A). Similarly pushing handle 112 (and thereby winch 146) along track 117 distally from the position shown in FIG. 1 moves the winch away from first site 60 (and towards second site 80), therefore reducing slack on tether 148 (FIG. 2B).

Delivery tool 110 (e.g., handle 112) can be adapted to control winch 146 in order to control the amount of tether 148 that is contained by/let out from the winch. For example, the handle can include a winch controller 114, operatively coupled to the winch, e.g., via a driveshaft that extends through shaft 116, such that operating the winch controller adjusts the amount of tether disposed within the heart (e.g., via rotation of the driveshaft such that more tether is wound/unwound from around spool 147).

In some implementations, delivery tool 110 (e.g., handle 112) is configured such that, during the advancement of winch 146 (and typically also ventricular anchor 144) away from first site 60 and towards second site 80, axial movement of the handle is limited in a manner that is linked to operation of winch 146, in order to ensure that tether 148 has sufficient but not excess slack.

For example, after tether 148 has been secured to first site 60 (e.g., by attaching leaflet anchor 142 to the leaflet), axial movement of handle 112 in a direction that moves ventricular anchor 144 towards second site 80 may be limited. Letting out some of tether 148 from winch 146 modifies the limit in a manner that allows further advancement in this direction. This is achieved by system 100 (e.g., delivery tool 110) comprising a limiter 124 that defines limits for axial movement of handle 112, and links these limits to the amount of the tether that has been let out from the winch.

In some implementations, this limiting is provided at least in part by handle 112 including a depth-stopper 120 that extends axially from the handle. Depth-stopper 120 can serve as at least a component of limiter 124. Handle 112 can be limited from moving axially by depth stopper 120 abutting (e.g., being obstructed by) part of mount 122. For example, mount 122 can comprise or define one or more stops 123, such as a distal stop 123a and a proximal stop 123b, which are positioned to obstruct depth stopper 120. FIG. 2A shows handle 112 being limited from being slid further proximally along track 117, due to depth-stopper 120 abutting proximal stop 123b of the mount. FIG. 2B shows handle 112 being limited from being slid further distally along track 117, due to depth-stopper abutting a distal stop 123a of the mount.

As well as winch controller 114 being operatively coupled to spool 147, it is also operatively coupled to depth-stopper 120, such that operation of the winch controller in a manner that operates the spool, also adjusts axial extension of the depth-stopper—and thereby adjusts the limit to which the handle is axially movable (e.g., with respect to mount 122).

For example, and as shown, winch controller 114 can be configured as a control knob, such that rotation of the control knob causes axial movement of depth-stopper 120 (e.g., via a rotary-to-linear translation between the control knob and the depth-stopper, such as via a worm drive). For example, handle 112 can be arranged such that rotating the knob in a manner that lets out tether 148 from around spool 147, also moves depth-stopper 120 proximally with respect to mount 122, thereby increasing an extent by which the handle (and therefore winch 146) can be advanced distally.

FIGS. 3A-B show that, in this manner, the spool can incrementally be advanced towards second site 80 while the operator is prevented from tugging/pulling on the tether at the first site, or from releasing too much tether within the heart. For example, FIG. 3A shows advancement of winch 146 from a position in which winch 146 was closer to first site 60 (e.g., from the position of the winch in FIG. 2B), to a position in which the winch is closer to the second site (FIG. 3A).

Inset A of FIG. 3A shows handle 112 in the same position as in FIG. 2B. Inset B of FIG. 3A shows spool controller (e.g., control knob) 114 being operated, thereby both (i) letting out (e.g., unwinding) tether 148 from around spool 147, and (ii) moving depth-stopper 120 proximally with respect to handle 112, e.g., as described hereinabove. This movement of depth-stopper 120 with respect to handle 112 allows for movement of the handle to a position more distal (with respect to mount 122) than was previously possible (inset C of FIG. 3A). For implementations in which mount 122 comprises proximal stop 123b, this proximal movement of depth-stopper 120, and therefore this letting out of tether 148, is limited by the depth stopper abutting the proximal stop. This may advantageously prevent the operator from generating too much slack in tether 148.

As shown in the main image of FIG. 3A, movement of handle 112 into this more distal position with respect to mount 122 moves winch 146 towards second site 80 within the heart. Thus, the movement of depth-stopper 120 with respect to handle 112 allows for movement of winch 146 towards second site 80 within the heart, e.g., until again limited by the depth stopper abutting distal stop 123a, as shown in inset C of FIG. 3A.

It is to be noted that operation of winch controller 114 can be considered to reconfigure limiter 124, modifying the limit, defined by the limiter, by which handle 112 is axially movable with respect to the mount.

FIG. 3B shows further advancement of winch 146 toward the second site (e.g., the main image of FIG. 3B shows the winch having arrived at the second site), by performing at least one more iteration of the above-mentioned steps (insets A-C). At this point, anchor 144 can be driven into tissue at the second site (e.g., tissue of ventricle 8). In some implementations, shaft 116 can comprise and/or ensheathe an anchor-control driveshaft (not shown) that operatively couples an anchor controller 111 (which, as shown, can be a control knob) of handle 112 to anchor 144, such that that operation of the anchor controller can drive (e.g., screw) the anchor into the tissue, e.g., with the anchor-control driveshaft transferring torque from anchor controller 111 to the anchor.

In some implementations, shaft 116 can be as described, mutatis mutandis, for shaft 810 and/or driveshaft subassembly 890 of International Patent Application No. PCT/IB2021/060436 (International Patent Publication No. WO2022/101817A2) which is incorporated herein by reference.

After optional further actuation of winch 146 to adjust tension and/or the length of tether 148 (not shown), shaft 116 can then be withdrawn from the heart, leaving implant 140 (e.g., upstream assembly 143 connected to downstream assembly 145 via tether 148) implanted within the heart. In some implementations, this further actuation is performed after operatively disabling limiter 124 (e.g., by operatively decoupling winch controller 114 from depth stopper 120, such as by operating a decoupling switch 115, or by moving/separating handle 112 and/or depth stopper 120 from mount 122).

In some implementations, the distal part of shaft 116 (e.g., the part of the shaft adapted to position implant 140 within the heart) can be telescopic, for example, having a distal portion and a proximal portion that are axially slidable with respect to each other. An example of such a telescopic shaft is described, mutatis mutandis, in International Patent Application PCT/IB2021/060436 (International Patent Publication No. WO2022/101817A2), which is incorporated herein by reference.

In some implementations in which shaft 116 is telescopic, handle 112 includes a shaft controller (e.g., a shaft extender), operatively coupled to the shaft such that operation of the shaft controller reversibly extends the distal portion of the shaft distally from the proximal portion of the shaft. Thus, in some implementations, advancement of winch 146 from first site 60 to second site 80 can be achieved by telescopically lengthening shaft 116 within the heart.

In some implementations, the shaft controller can be operatively coupled to winch controller 114 in a manner that limits telescopic lengthening according to an amount of tether 148 let out from the winch-thereby defining a limiter having similar functionality to that described hereinabove, albeit without a depth stopper.

In some implementations, the operative coupling between the shaft controller and winch controller 114 can be such that operating the shaft controller to lengthen the shaft within the heart (e.g., such that the winch moves towards the second site) automatically operates the winch controller (e.g., such that more tether 148 is exposed within the heart). Such coupling between the shaft controller and winch controller 114 may automatically ensure that tether 148 is let out at the rate required by the movement of winch 146, thus preventing excess slack in the tether and/or tugging of the tether at first site 60.

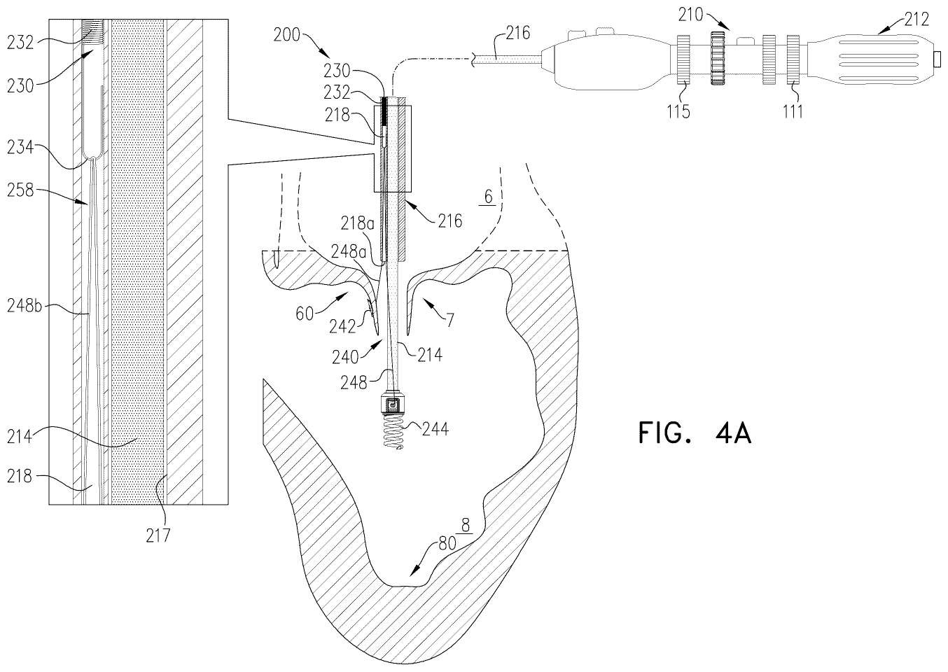

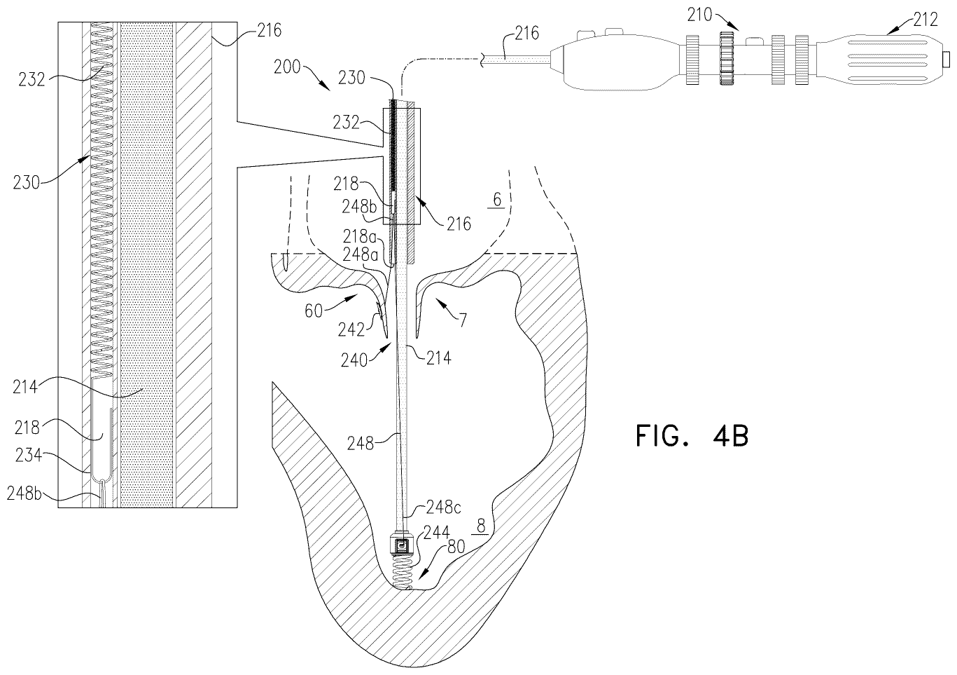

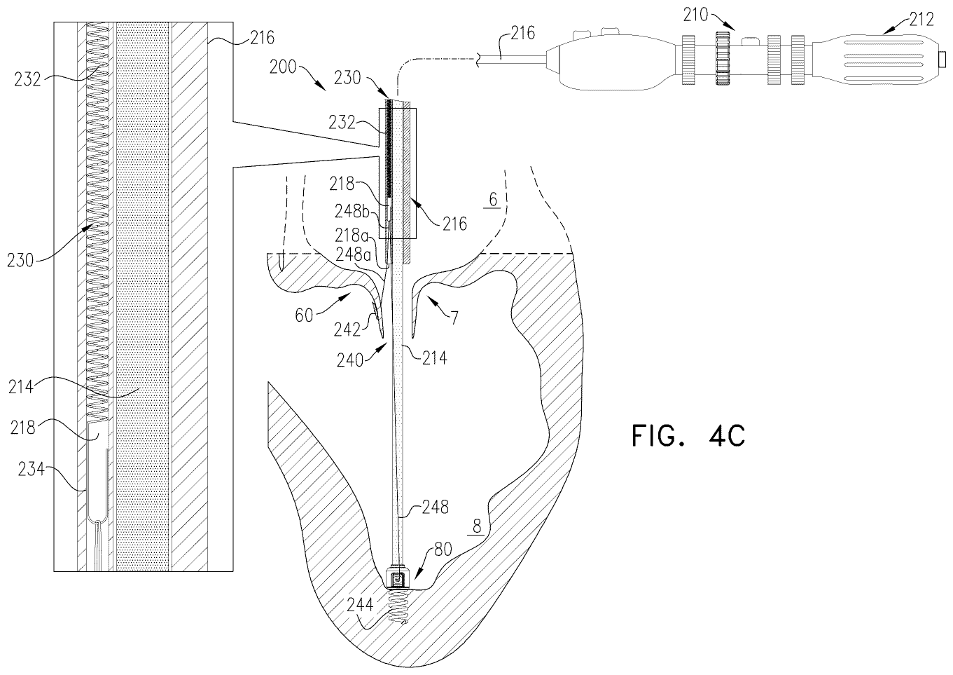

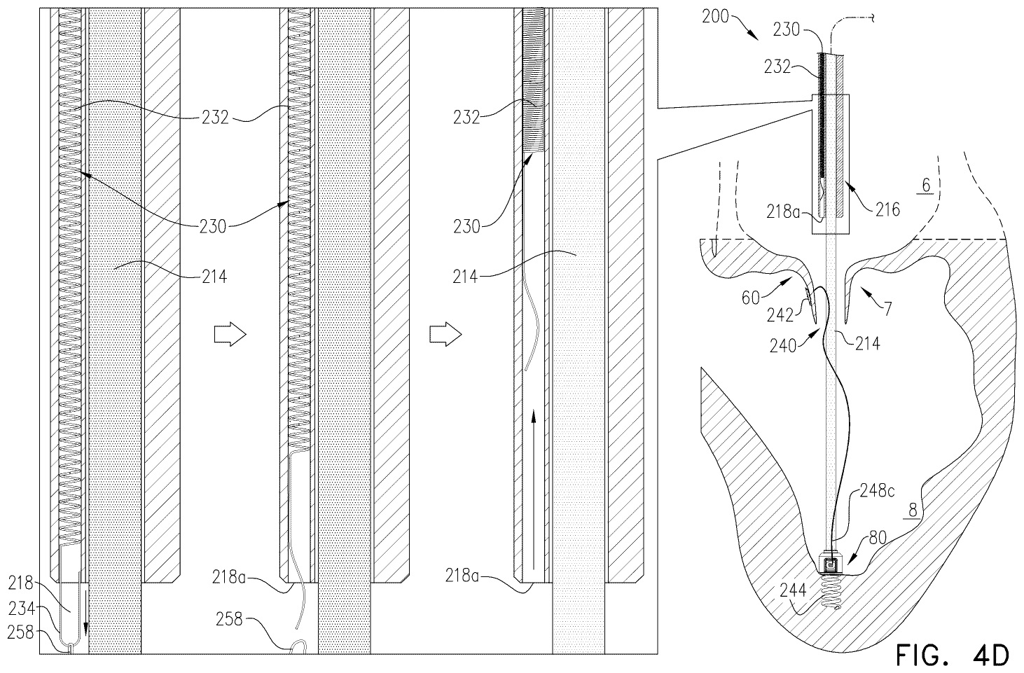

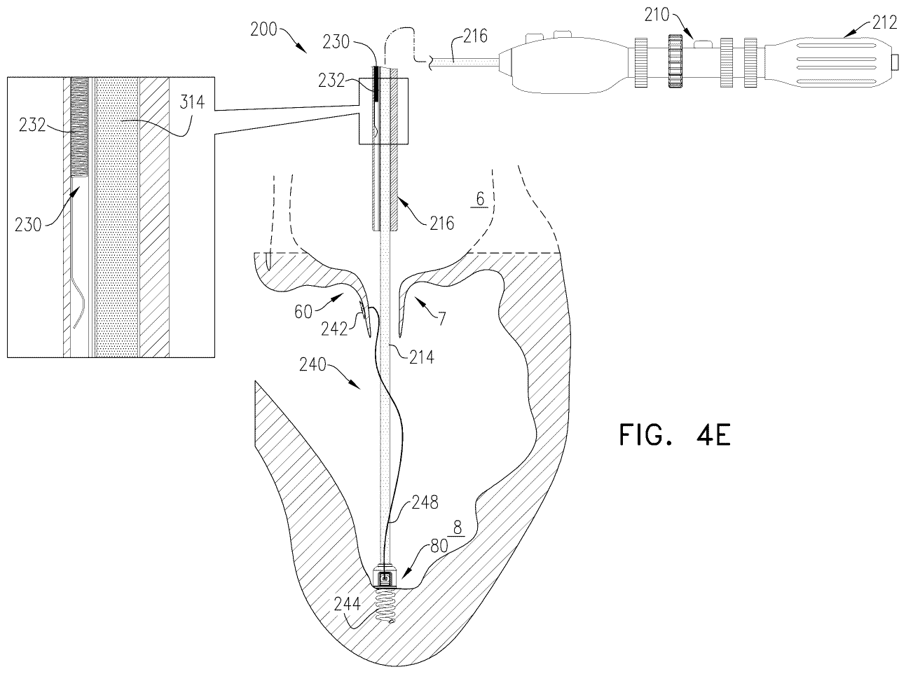

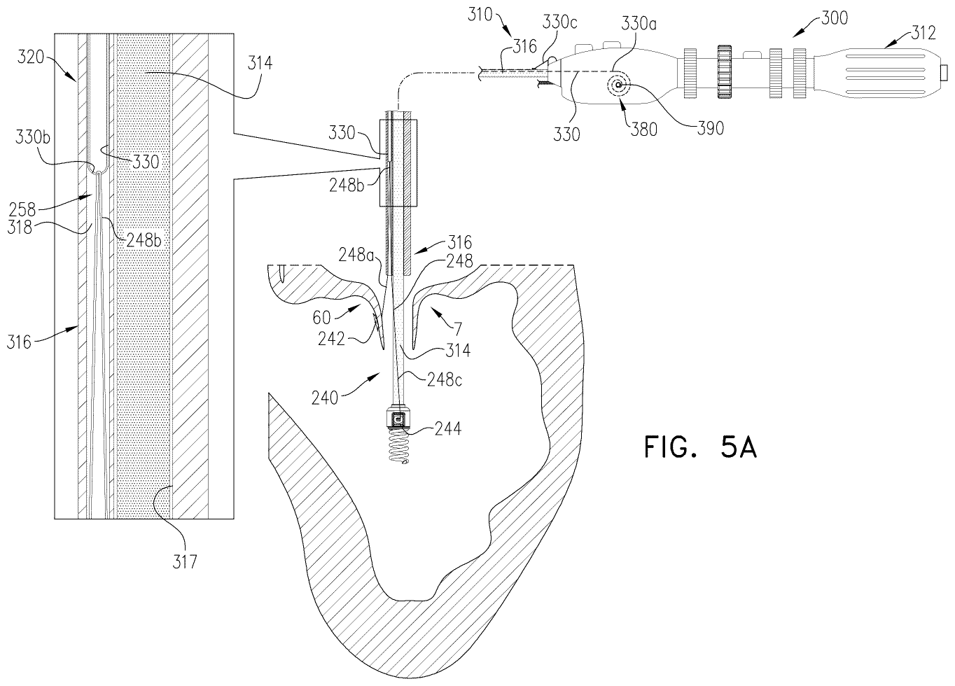

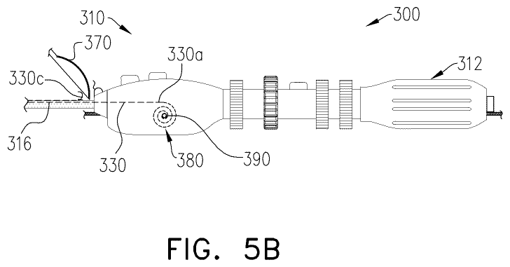

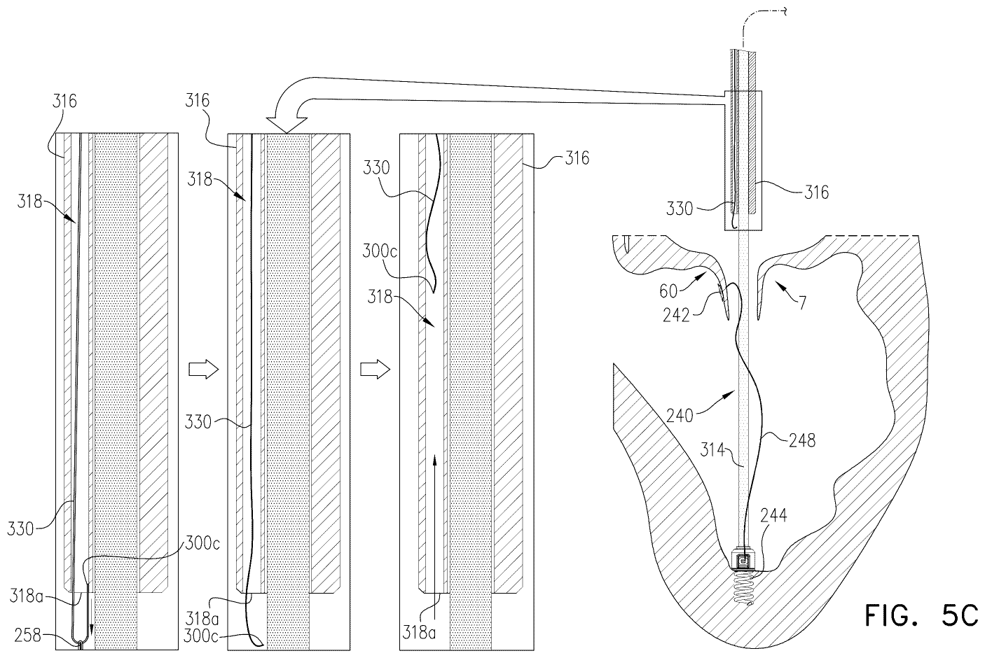

Reference is now made to FIGS. 4A-E and 5A-C, which are schematic illustrations of systems and techniques for controlling excess slack in a tether of an implant during implantation of the implant. For example, and similarly to the methods and systems described hereinabove, the current techniques can be used for preventing excess slack in a tether 248 during advancement of part (e.g., an end portion 248c thereof) of the tether from a first site within the heart (e.g., first site 60) towards a second site within the heart (e.g., second site 80), e.g., in order to reduce a likelihood of tangling and/or ensnarement of the tether. Similarly, devices and techniques described can be used to reduce a likelihood of the tether tugging on, or becoming disconnected from, the first site. The methods and techniques can be performed on a living animal or on a simulation, such as on a cadaver, cadaver heart, simulator (e.g., with the body parts, heart, tissue, etc. being simulated), etc.

FIGS. 4A-E illustrate a system 200 comprising an implant 240, and a delivery tool 210 for use with the implant, in accordance with some implementations. In some implementations, and as illustrated in FIGS. 4A-E, implant 240 can be considered to be a variant of implant 140 (or substantially identical to implant 140) described hereinabove. For example, implant 240 can comprise an upstream assembly comprising a leaflet anchor 242 (which can be considered to be a variant of leaflet anchor 142), a downstream assembly comprising a ventricular anchor 244 (which can be considered to be a variant of ventricular anchor 144), and a tether (e.g., tether 248) extending therebetween. In some implementations, implant 240 can differ from implant 140 by not comprising a winch.

In some implementations, implant 240 does not comprise an anchor 242 (e.g., a leaflet anchor at one end of tether 248). In some implementations, implant 240 does not comprise an anchor 244 (e.g., a ventricular anchor at another end of tether 248). For example, one or both ends of the tether of implant 240 can be attached to its respective site by stitching. For example, implant 240 can consist substantially of a tether (e.g., tether 248, or a suture) for securing at the first and second sites of a heart by stitching.

In some implementations, implant 240 can be an annuloplasty implant, for example an annuloplasty ring structure.

In some implementations, delivery tool 210 is provided and configured for delivery, positioning, and implantation of implant 240. Tool 210 comprises an extracorporeal handle 212, coupled to a flexible tube 216 (e.g., a catheter or sheath), which extends from the handle to a distal part of the tool at which implant 240 is disposed (e.g., to which implant 240 is coupled). Tube 216 is shaped to define a lumen 217 therethrough. During delivery and/or implantation of implant 240, at least part of implant 240 can be coupled to and/or disposed within a distal part of tube 216. Delivery tool 210 further comprises a channel 218. In some implementations, and as shown, channel 218 can be a lumen (e.g., a secondary lumen) of tube 216. In some implementations, channel 218 can be, or can be defined by, an additional component (e.g., an additional tube/channel) of the delivery tool. In some implementations, delivery tool 210 comprises a shaft 214 for delivery and implantation of anchor 244 within the heart. Shaft 214 can be considered to be a variant of, or can be substantially identical to, shaft 116, mutatis mutandis, for example, shaft 214 can comprise and/or ensheathe a driveshaft for driving anchor 244 into the tissue.

Shaft 214 can extend through tube 216 (e.g., through lumen 217 as shown), or can extend alongside the tube.

A portion of tether 248 (e.g., a middle portion 248b) can be confined within channel 218. In some implementations, and as shown in FIGS. 4A-C, after securement of tether 248 (e.g., a first end portion 248a thereof) to a first site 60 (e.g., at a leaflet of a valve), the tether is released progressively from channel 218 as a second end portion 248c of the tether is advanced away from the first site and towards the second site. For example, middle portion 248b of the tether can extend, as a bight 258, from portion 248a, into channel 218, and back out of the channel towards end portion 248c. It is hypothesized that confining middle portion 248b, and controlling/limiting its release, may prevent tangling and/or ensnarement of the tether within the heart during the advancement and/or implantation of implant 240 (e.g., during advancement and/or implantation of ventricular anchor 244 thereof).

In some implementations, this confinement is achieved by a retaining member 230. As illustrated in FIGS. 4A-D, a portion (e.g., an end portion) of the retaining member can be constrained to define a hook 234 by the inner wall of the channel 218, the hook being hooked to bight 258 of tether 248, thereby retaining middle portion 248b within the channel.

Part of retaining member 230 can be fixed with respect to channel 218. For example, part of the retaining member can be fixed with respect to the channel at an attachment site, such that progressively pulling the tether out of the opening of the channel pulls the tether away from the attachment site and out of the opening of the channel.

In some implementations, this attachment can be provided by a proximal portion of the retaining member being fixed and/or attached to the inner wall of the channel and/or to a proximal part of the channel. For implementations in which channel 218 is defined by and/or fixed to tube 216, the part of the retaining member can be fixed to the tube.

In some implementations, retaining member 230 can include a deformable portion 232 that is deformable such that pulling tether 248 (e.g., as a result of moving end portion 248c of the tether toward second site 80) can pull hook 234 towards a distal opening 218a of channel 218, thereby increasing the amount of tether 248 exposed within the heart (e.g., exposed out of opening 218a). That is, and as is illustrated by the transition from FIG. 4A to FIG. 4C, progressive deformation of retaining member 230 allows tether 248 to be progressively released from channel 218 as it is pulled.

In some implementations, once a predetermined amount of pulling has occurred, hook 234 becomes exposed out of opening 218a, and once it is no longer constrained by the inner wall of channel 218, the hook responsively opens, thereby unhooking from the bight (e.g., the hook is pulled open by the tether).

The predetermined amount of pulling at which the hook becomes exposed may be dependent on mechanical characteristics of deformable portion 232 (e.g., its deformability, e.g., stretchability and/or elasticity, such as spring constant) and/or on the dimensions (e.g., lengths) of retaining member 230 and channel 218.

FIGS. 4A-E represent a series of example steps that can be performed by an operator during implantation of implant 240 within the heart. Subsequently to end portion 248a being attached to first site 60 (e.g., subsequently to leaflet anchor 242 being anchored to the leaflet), end portion 248c (and anchor 244, if present) of tether 248 is advanced, from a position in which the anchor is closer to the first site (FIG. 4A), away from the first site (e.g., away from leaflet anchor 242), towards second site 80 (FIG. 4B), while portion 248b of the tether remains confined within channel 218. This pulls portion 248b and therefore retaining member 230 (e.g., hook 234 thereof) towards distal opening 218a of channel 218, the pulling being facilitated by deformation of deformable portion 232 (as can be seen in the transition between FIGS. 4A-B).

In some implementations, system 200 is configured to facilitate and/or ensure that anchor 244 is implanted within tissue of the heart (e.g., into tissue of ventricle 8) while hook 234 remains hooked onto bight 258 and/or remains disposed within channel 218 (FIG. 4C).

Alternatively, in some implementations, system 200 can be configured to facilitate and/or ensure that bight 258 is released from retaining member 230 prior to anchor 244 being implanted within the tissue (e.g., the bight is released once it has been determined that anchor 244 is disposed at the second site, prior to anchoring of the anchor).

Once it has been determined that tether 248 can be released from channel 218 (e.g., once it has been determined that implant 240 has been successfully implanted), the operator may simply withdraw tube 216 (and optionally also shaft 214) from the subject. As shown in FIG. 4D, as tube 216 is withdrawn, hook 234 becomes pulled (by tether 248) out of distal opening 218a, such that it is no longer constrained by the inner wall of channel 218, therefore responsively opening and unhooking from bight 258 (e.g., the hook is pulled open by the tether).

At this point, for implementations in which implant 240 includes a winch at anchor 244, shaft 214 and/or a driveshaft disposed therewithin may remain temporarily coupled to the downstream assembly in order to operate the winch.

In some implementations, deformable portion 232 of retaining member 230 is elastically deformable, such that if tether 248 is pulled, but not to the predetermined amount of pulling (e.g., such that bight 258 of the tether remains hooked by hook 234 within channel 218, as in FIGS. 4A-C), relaxing of the pulling results in the retaining member drawing the tether back into channel 218. For example, and as shown, retaining member 230 (e.g., deformable portion 232 thereof) can be a spring (e.g., an extension spring). Thus, relaxing tension on tether 248 allows deformable portion 232 to transition from a pulled state (e.g., as shown in FIG. 4B) back to a more relaxed state (e.g., as shown in FIG. 4A). Such an elastically-deformable portion may allow repeated repositioning of the end of tether 248 (and anchor 244, if present) within the heart, while maintaining tension on the tether (e.g., a small amount of tension, sufficient to reduce slack without interfering with the repositioning).

In some implementations, deformable portion 232 of retaining member 230 is plastically deformable.

In some implementations, a single elongate element (e.g., a wire) is shaped to define both deformable portion 232 (e.g., a spring) and hook 234.

FIGS. 5A-C illustrate a system 300 comprising implant 240 and a delivery tool 310 for use with the implant, in accordance with some implementations. System 300 can be considered to be a variant of system 200, mutatis mutandis, e.g., comprising tool 310 instead of tool 210.

Tool 310 comprises an extracorporeal handle 312, coupled to a flexible tube 316 (e.g., a catheter or sheath), which extends from the handle to a distal part of the tool at which implant 240 is disposed (e.g., to which implant 240 is coupled). Tube 316 is shaped to define a lumen 317 therethrough. During delivery and/or implantation of implant 240, at least part of implant 240 can be coupled to and/or disposed within a distal part of tube 316. Delivery tool 310 further comprises a channel 318, which can be a lumen (e.g., a secondary lumen) of tube 316, or can be an additional component (e.g., an additional catheter/channel) of the delivery tool. In some implementations, delivery tool 310 comprises a shaft 314 for delivery and implantation of anchor 244 within the heart. Shaft 314 can be considered to be a variant of, or can be substantially identical to, shaft 116 and/or shaft 214, mutatis mutandis, for example, shaft 314 can comprise and/or ensheathe a driveshaft for driving anchor 244 into the tissue.

Shaft 314 can extend through tube 316 (e.g., as shown), or can extend alongside the tube.

Similarly to system 200, a portion of the tether remains confined within channel 318 (which can be a lumen of tube 316 and/or fixed to the tube) after securement of tether (e.g., a first end portion 248a thereof) to a first site 60 (e.g., at a leaflet), and the tether can be released progressively from the channel as a second end portion 248c is advanced to the second site. For example, a middle portion 248b of tether 248 can extend, as a bight 258, into channel 318, and back out of the channel, and towards end portion 248c of the tether. Confining this middle portion 248b of the tether may advantageously prevent tangling and/or ensnarement of the tether within the heart during the advancement and/or implantation of implant 240 (e.g., during advancement and/or implantation of ventricular anchor 244 thereof).

In contrast to confining tether 248 in the channel using a hook 234 as described hereinabove with respect to system 200, FIGS. 5A-C show the confinement of tether 248 within channel 318 being provided by a retaining member 320 that is defined by an elongate member 330 (e.g., a thread, a suture, a line, or a wire) that extends distally through the channel, loops around bight 258 of the tether (e.g., elongate member 330 forms a loop 330b around the bight), and returns proximally through the channel (FIG. 5A).

In some implementations, one or both ends (330a, 330c) of elongate member 330 are disposed at a proximal portion of system 300 that is configured to remain outside of the subject (e.g., by being coupled to handle 312). Pulling tether 248 (e.g., end portion 248c of the tether) within the heart pulls loop 330b towards a distal opening 318a of the channel, increasing the amount of tether exposed from the channel within the heart.

In some implementations, an end 330a of elongate member 330 is coupled to a deformable member 380 (e.g., that can be a variant, or substantially identical to, deformable portion 232, mutatis mutandis), e.g., in order to maintain tension on tether 248 during implantation of implant 240 (e.g., a small amount of tension, sufficient to reduce slack). In the example shown, deformable member 380 is a torsion spring.

In some implementations, once it has been determined that tether 248 can be released from channel 318 (e.g., once it has been determined that implant 240 has been successfully implanted), loop 330b can be released from around bight 258. For example, and as illustrated in FIGS. 5B-C, this releasing can be performed by cutting one end of the elongate member (FIG. 5B), e.g., using a scalpel 370, and subsequently pulling the other end of the elongate member such that loop 300b can be withdrawn from around bight 258, and out of the subject (FIG. 5C). In the example shown, the end that is cut is end 330c, e.g., the end that is not coupled to deformable member 380. However, in some implementations, end 330a can be cut. Whichever end is to be cut, tool 310 can be configured such that the to-be-cut end is exposed at an extracorporeal proximal portion of the tool (e.g., from handle 312), e.g., such that it is presented to and/or accessible by the operator for convenient cutting.

In some implementations, bight 258 is released from elongate member 330 prior to the tether being secured to second site 80 (e.g., prior to anchor 244 being anchored to the tissue at the second site). In some implementations bight 258 is released from elongate member 330 subsequently to the tether being secured to second site 80 (e.g., prior to anchor 244 being anchored to the tissue at the second site).

In some implementations, tool 310 comprises a bobbin 390 (e.g., mounted on handle 312), such that elongate member 330 can be withdrawn from channel 318 by winding the elongate member around the bobbin. Bobbin 390 can be coupled to a key or knob via which the operator can rotate the bobbin, in order to pull and unloop the elongate member from bight 258 of tether 248.

In some implementations, such as some implementations in which deformable member 380 comprises a torsion spring, the deformable member can be disposed circumferentially around bobbin 390, e.g., as shown. In some implementations, deformable member 380 can rotate with bobbin 390.

In some implementations, such as some implementations in which deformable member 380 comprises a torsion spring, the deformable member can be disposed inside of bobbin 390. In some implementations, deformable member 380 can rotate with bobbin 390.

Examples (some non-limiting examples of the concepts herein are recited below):

Example 1. A system comprising: (A) an implant, comprising a winch, comprising a spool, and a tether, coupled to the spool; and (B) a delivery tool, configured to transluminally deliver the implant to a heart, and comprising: (i) a shaft, coupled to the winch, and configured to transluminally position the winch within the heart, (ii) a handle, comprising a winch controller, and coupled to a proximal part of the shaft such that distal movement of the handle moves the winch distally into the heart, the proximal part of the shaft defining an axis of the delivery tool, and/or (iii) a depth-stopper, extending axially from the handle; wherein the winch controller is operatively coupled to the winch and to the depth-stopper such that operation of the winch controller in a manner that adjusts an amount of winding of the tether around the spool, also adjusts axial extension of the depth-stopper from the handle.

Example 2. The system according to example 1, wherein the depth-stopper comprises a rod.

Example 3. The system according to any one of examples 1-2, wherein the depth-stopper extends parallel to the axis.

Example 4. The system according to any one of examples 1-3, wherein the shaft is configured to transluminally position the winch within the heart while extending, from the handle, through vasculature of a subject, to the winch within the heart.

Example 5. The system according to any one of examples 1-4, wherein the winch controller comprises a control knob, and is operatively coupled to the winch and to the depth-stopper such that rotation of the control knob in a manner that adjusts an amount of winding of the tether around the spool, also adjusts axial extension of the depth-stopper from the handle.

Example 6. The system according to example 5, wherein the winch controller is operatively coupled to the winch and to the depth-stopper via a rotary-to-linear mechanism.

Example 7. The system according to example 6, wherein the rotary-to-linear mechanism comprises a worm drive.

Example 8. The system according to any one of examples 1-7, wherein the implant comprises an anchor, coupled to the winch, and configured to anchor the winch to tissue at a site in the heart.

Example 9. The system according to example 8, wherein (i) the anchor is a second anchor, (ii) the site is a second site, and (iii) the implant further comprises a first anchor, coupled to a first end portion of the tether, and configured to anchor the first end portion of the tether to tissue at a first site within the heart, and (iv) the shaft is configured to transluminally advance the winch and the second anchor towards the second site while the first anchor remains anchored at the first site.

Example 10. The system according to example 9, wherein (i) the first site is a leaflet site at a leaflet of a valve of the heart, (ii) the first anchor is a leaflet anchor adapted to be anchored to the leaflet, (iii) the second site is a ventricular site in a ventricle downstream of the valve, (iv) the second anchor is a ventricular anchor, and (v) the shaft is configured to transluminally advance the winch and the ventricular anchor towards the ventricular site while the first anchor remains anchored at the leaflet site.

Example 11. The system according to any one of examples 1-10, wherein the delivery tool has a state in which the depth-stopper extends distally from the handle.

Example 12. The system according to example 11, wherein the winch controller is operatively coupled to the winch and to the depth-stopper such that operation of the winch controller in a manner that decreases the amount of winding of the tether around the spool, also moves the depth-stopper proximally with respect to the handle.

Example 13. The system according to example 12, wherein the winch controller is operatively coupled to the winch and to the depth-stopper such that operation of the winch controller in a manner that increases the amount of winding of the tether around the spool, also moves the depth-stopper distally with respect to the handle.

Example 14. The system according to example 11, wherein the winch controller is operatively coupled to the winch and to the depth-stopper such that operation of the winch controller in a manner that decreases the amount of winding of the tether around the spool, also moves the depth-stopper distally with respect to the handle.

Example 15. The system according to example 14, wherein the winch controller is operatively coupled to the winch and to the depth-stopper such that operation of the winch controller in a manner that increases the amount of winding of the tether around the spool, also moves the depth-stopper proximally with respect to the handle.

Example 16. The system according to example 11, wherein the state is a first state, and wherein the delivery tool has a second state in which the depth-stopper extends proximally from the handle.

Example 17. The system according to example 11, wherein, in the state, the depth-stopper extends from the handle simultaneously distally and proximally.

Example 18. The system according to any one of examples 1-17, further comprising a mount in which the handle is mountable, and which, while the handle is mounted in the mount, is configured to obstruct axial movement of the depth-stopper along an axial path by being disposed in the axial path.

Example 19. The system according to example 18, wherein the mount extends perpendicular to the axis.

Example 20. The system according to example 18, wherein the winch controller is operatively coupled to the winch and to the depth-stopper such that: (i) operation of the winch controller in a manner that decreases an amount of winding of the tether around the spool, also moves the depth-stopper proximally with respect to the mount, and/or (ii) operation of the winch controller in a manner that increases an amount of winding of the tether around the spool, also moves the depth-stopper distally with respect to the mount.

Example 21. The system according to example 18, wherein the winch controller is operatively coupled to the winch and to the depth-stopper such that: (i) operation of the winch controller in a manner that increases an amount of winding of the tether around the spool, also moves the depth-stopper proximally with respect to the mount, and/or (ii) operation of the winch controller in a manner that decreases an amount of winding of the tether around the spool, also moves the depth-stopper distally with respect to the mount.

Example 22. The system according to example 18, wherein the winch controller is operatively coupled to the winch and to the depth-stopper such that operation of the winch controller in the manner that adjusts the amount of winding of the tether around the spool, also adjusts an axial position of the depth-stopper with respect to the mount.

Example 23. The system according to example 22, wherein the mount comprises: (i) a distal stop that, while the handle is mounted in the mount, is positioned to obstruct movement of the depth-stopper distally by being disposed in the axial path; and/or (ii) a proximal stop that, while the handle is mounted in the mount, is positioned to obstruct movement of the depth-stopper proximally by being disposed in the axial path.

Example 24. The system according to example 23, wherein the winch controller is operatively coupled to the winch and to the depth-stopper such that: (i) operation of the winch controller in a manner that decreases the amount of winding of the tether around the spool, also moves the depth-stopper axially away from the distal stop, and/or (ii) operation of the winch controller in a manner that increases the amount of winding of the tether around the spool, also moves the depth-stopper axially toward the distal stop.

Example 25. The system according to example 23, wherein the winch controller is operatively coupled to the winch and to the depth-stopper such that: (i) operation of the winch controller in a manner that decreases the amount of winding of the tether around the spool, also moves the depth-stopper axially toward the distal stop, and/or (ii) operation of the winch controller in a manner that increases the amount of winding of the tether around the spool, also moves the depth-stopper axially away from the distal stop.

Example 26. A system comprising: (A) an implant, comprising a spool and a tether, coupled to the spool; and (B) a delivery tool, configured to transluminally deliver the implant to a heart, and comprising: (i) a shaft, coupled to the spool, and configured to transluminally position the spool within the heart, (ii) a mount, and (iii) a handle, mountable on the mount, and coupled to a proximal end of the shaft such that axial movement of the handle with respect to the mount axially moves the shaft with respect to the mount, wherein the delivery tool comprises a limiter, configured to define a limit by which the handle is axially movable with respect to the mount, and/or wherein the handle comprises a controller that operatively couples the spool to the limiter such that operation of the controller in a manner that adjusts an amount of winding of the tether around the spool, also modifies the limit.

Example 27. The system according to example 26, wherein the handle is mountable on the mount subsequently to the shaft transluminally positioning the spool within the heart.

Example 28. The system according to any one of examples 26-27, wherein the limiter comprises a depth-stopper.

Example 29. The system according to any one of examples 26-28, wherein: (i) the delivery tool comprises a shaft controller that is adapted to lengthen the shaft within the heart, and/or (ii) operating the shaft controller to lengthen the shaft within the heart automatically operates the controller to increase the amount of tether exposed within the heart.

Example 30. The system according to any one of examples 26-29, wherein the shaft is configured to transluminally position the spool within the heart while extending, from the handle, through vasculature of a subject, to the spool within the heart.

Example 31. The system according to any one of examples 26-30, wherein the controller comprises a control knob, and is operatively coupled to the spool and to the limiter such that rotation of the control knob adjusts the amount of winding of the tether around the spool, and also modifies the limit.

Example 32. The system according to any one of examples 26-31, wherein the controller is operatively coupled to the spool and to the limiter via a rotary-to-linear mechanism.

Example 33. The system according to example 32, wherein the rotary-to-linear mechanism comprises a worm drive.

Example 34. The system according to any one of examples 26-33, wherein the implant comprises an anchor, coupled to the spool, and configured to anchor the spool to tissue at a site in the heart.

Example 35. The system according to example 34, wherein the anchor is a second anchor, the site is a second site, and the implant further comprises a first anchor, coupled to a first end portion of the tether, and configured to anchor the first end portion of the tether to tissue at a first site within the heart, and the shaft is configured to transluminally advance the spool and the second anchor towards the second site while the first anchor is anchored at the first site.

Example 36. The system according to example 35, wherein the first site is a leaflet site at a leaflet of a valve of the heart, the first anchor is adapted to be anchored to the leaflet site, the second site is a ventricular site in a ventricle downstream of the valve, the second anchor is adapted to be anchored to the ventricular site, and/or the shaft is configured to transluminally advance the spool and the second anchor towards the ventricular site while the first anchor is anchored at the leaflet site.

Example 37. The system according to any one of examples 26-36, wherein, while the handle is mounted on the mount, the mount is disposed in the axial path such that the mount obstructs the axial movement of the handle upon the handle reaching the limit.

Example 38. The system according to example 37, wherein the mount comprises: (i) a distal stop that, while the handle is mounted on the mount, is positioned to obstruct movement of the limiter distally by being disposed in the axial path; and/or (ii) a proximal stop that, while the handle is mounted on the mount, is positioned to obstruct movement of the limiter proximally by being disposed in the axial path.

Example 39. A method comprising (i) using a delivery tool having a handle, transluminally advancing, to a heart, a winch operatively coupled to a tether, (ii) attaching the tether to tissue at a first site within the heart, (iii) operating a winch controller of the handle to actuate the winch and move, in a first axial direction with respect to the handle, a depth-stopper that extends axially from the handle, and (iv) while the tether remains coupled to the tissue at the first site, advancing the winch away from the first site by advancing the handle of the delivery tool in a second axial direction that is opposite to the first direction.

Example 40. The method according to example 39, wherein advancing the winch away from the first site comprises advancing the winch away from the first site subsequently to operating the winch controller to actuate the winch.

Example 41. The method according to any one of examples 39-40, further comprising mounting the handle in a mount.

Example 42. The method according to example 41, wherein mounting the handle in the mount comprises mounting the handle in the mount subsequently to the step of transluminally advancing the winch to the heart, and prior to the step of operating the winch controller.

Example 43. The method according to example 41, wherein operating the winch controller comprises operating the winch controller to move the depth-stopper in the first axial direction with respect to the handle such that the depth-stopper approaches a stop defined by the mount.

Example 44. The method according to example 43, wherein operating the winch controller comprises operating the winch controller until the depth-stopper abuts the stop.

Example 45. The method according to example 41, wherein advancing the handle of the delivery tool in the second axial direction comprises advancing the handle of the delivery tool in the second axial direction such that the depth-stopper approaches a stop defined by the mount.

Example 46. The method according to example 45, wherein advancing the handle of the delivery tool in the second axial direction comprises advancing the handle of the delivery tool in the second axial direction until the depth-stopper abuts the stop.

Example 47. The method according to any one of examples 39-46, wherein the steps of operating the winch controller and advancing the winch are performed iteratively at least until the winch reaches a second site within the heart.

Example 48. The method according to example 47, further comprising anchoring the winch to tissue at the second site.

Example 49. The method according to example 48, further comprising, subsequently to anchoring the winch to the tissue at the second site, adjusting an amount of the tether that is let out from the winch by operating the winch controller to actuate the winch.

Example 50. The method according to example 48, further comprising, subsequently to anchoring the winch to the tissue at the second site, adjusting tension on the tether by operating the winch controller to actuate the winch.

Example 51. A system for controlling slack in a tether during implantation of the tether in a heart of a subject, the system comprising: (A) the tether, the tether having a first end portion, a second end portion, and a bight therebetween, and (B) a delivery tool, configured to secure the first end portion of the tether and the second end portion of the tether within the heart, and comprising: (i) a channel having an inner wall and a distal opening, and (ii) a retaining member, constrained by the inner wall to define a hook within the channel, wherein: (1) the bight of the tether is confined within the channel by being hooked onto the hook; (2) the retaining member is deformable responsively to pulling of the tether out of the distal opening in a manner that moves the hook toward the distal opening, and (3) the retaining member is configured such that, upon the hook exiting the channel via the distal opening, the hook responsively opens and unhooks from the bight.

Example 52. The system according to example 51, wherein the retaining member comprises a deformable portion that is plastically deformable responsively to the pulling of the tether.

Example 53. The system according to any one of examples 51-52, wherein: (i) the delivery tool comprises a shaft, and an extracorporeal handle coupled to a proximal part of the shaft, and/or (ii) a distal part of the shaft is coupled to the tether such that, while the shaft is disposed through vasculature of the subject to the tether within the heart, the shaft is configured to transluminally position the tether within the heart by pulling the tether out of the distal opening in the manner that moves the hook toward the distal opening.

Example 54. The system according to example 53, wherein: (i) the delivery tool comprises a tube defining the channel, and (ii) the shaft extends through the tube and is configured to transluminally position the tether within the heart by the shaft sliding distally outwards from the tube in a manner that pulls the tether distally out of the distal opening.

Example 55. The system according to example 54, wherein the handle is coupled to a proximal part of the tube, and wherein the handle is configured to slide the shaft distally outward from the tube.

Example 56. The system according to any one of examples 51-55, wherein the retaining member comprises a deformable portion that is elastically deformable responsively to the pulling of the tether and is adapted to draw the tether back into the channel, responsively to relaxing of the pulling of the tether.

Example 57. The system according to example 56, wherein the deformable portion comprises a spring.

Example 58. The system according to example 56, wherein the retaining member comprises a unitary elongate member that is shaped to define the deformable portion and the hook.

Example 59. The system according to any one of examples 51-58, wherein: (A) the system comprises an implant that comprises the tether, a first component, and a second component, the tether tethering the first component to the second component, (B) the delivery tool is configured to: (i) anchor the first component at a first site within the heart, and (ii) subsequently, advance the second component from the first site toward a second site within the heart while (a) the first component remains anchored at the first site, and (b) the bight of the tether remains confined within the channel by the hook of the retaining member.

Example 60. The system according to example 59, wherein the second component comprises a winch, operatively coupled to the tether.

Example 61. The system according to example 59, wherein: (i) the first site is a leaflet site at a leaflet of a valve of the heart, (ii) the first component is a leaflet anchor adapted to be anchored to the leaflet, (iii) the second site is a ventricular site in a ventricle downstream of the valve, (iv) the second component is a ventricular anchor adapted to be anchored to ventricular tissue, and/or (v) the delivery tool is configured to transluminally advance the ventricular anchor towards the ventricular site while the first component remains anchored at the leaflet site.

Example 62. A method, the method comprising: (i) using a delivery tool that defines a channel at a distal portion thereof, advancing a tether to a heart while a bight of the tether is retained within the channel by a hook disposed in the channel; (ii) while the distal portion of the delivery tool is disposed within the heart, progressively pulling the tether out of an opening of the channel such that the hook moves towards the opening of the channel; and (iii) subsequently, releasing the bight from the hook by pulling the tether sufficiently to pull the hook out of the opening such that the hook responsively unhooks from the bight.

Example 63. The method according to example 62, wherein: (i) the hook is a component of a retaining member that further comprises a deformable portion coupled to the hook, and/or (ii) progressively pulling the tether out of the opening of the channel comprises progressively pulling the tether out of the opening of the channel such that the deformable portion deforms as the hook moves towards the distal opening.

Example 64. The method according to example 63, wherein part of the retaining member is fixed with respect to the channel at an attachment site, and wherein progressively pulling the tether out of the opening of the channel comprises progressively pulling the tether away from the attachment site and out of the opening of the channel.

Example 65. The method according to any one of examples 62-64, wherein pulling the tether sufficiently to pull the hook out of the opening comprises withdrawing the delivery tool proximally away from the tether while the tether remains within the heart.

Example 66. The method according to example 62, wherein the tether is a component of an implant, the implant further includes an anchor coupled to the tether, and the method further comprises anchoring the anchor to tissue of the heart.

Example 67. The method according to example 66, wherein anchoring the anchor to tissue of the heart comprises anchoring the anchor to tissue of the heart subsequently to unhooking the hook from the bight.

Example 68. The method according to example 66, wherein anchoring the anchor to tissue of the heart comprises anchoring the anchor to tissue of the heart prior to unhooking the hook from the bight.

Example 69. The method according to example 66, wherein the implant further includes a winch, and wherein anchoring the anchor comprises anchoring the winch to tissue by anchoring the anchor.

Example 70. The method according to example 69, further comprising, subsequently to anchoring the winch to the tissue, adjusting an amount of the tether that is let out from the winch by actuating the winch.

Example 71. The method according to example 69, further comprising, subsequently to anchoring the winch to the tissue, adjusting tension on the tether by actuating the winch.

Example 72. The method according to example 66, wherein the anchor is a second anchor, the implant further including a first anchor coupled to the tether, and anchoring the anchor comprises anchoring the second anchor at a second site within the heart, such that the tether extends from the second anchor, anchored at the second site, to the first anchor.

Example 73. The method according to example 72, further comprising anchoring the first anchor at a first site within the heart, prior to anchoring the second anchor at the second site.

Example 74. The method according to example 73, wherein progressively pulling the tether out of the opening of the channel comprises progressively pulling the tether out of the opening of the channel by advancing the second anchor away from the first site toward the second site, subsequently to anchoring the first anchor.

Example 75. A system for controlling slack in a tether of an implant during implantation of the tether in a heart of a subject, the system comprising: (i) the implant, comprising the tether, the tether having a first end portion, a second end portion, and a bight therebetween; and (ii) a delivery tool, configured to secure the first end portion of the tether and the second end portion of the tether within the heart, and comprising: (A) an extracorporeal control portion; (B) a shaft extending, from the extracorporeal control portion to the implant, and adapted to position the implant within the heart; (C) at a distal part of the shaft, a channel having a distal opening; and/or (D) an elongate member, coupled to the extracorporeal control portion, and extending away from the extracorporeal control portion to form a loop within the channel; wherein the system has a delivery state in which: (i) the bight of the tether is disposed within the channel, (ii) the first and second end portions of the tether are disposed outside of the distal opening of the channel, (iii) the elongate member confines the bight of the tether within the channel by being looped onto the bight, and/or (iv) at least a part of the elongate member is decouplable from the extracorporeal control portion to facilitate release of the bight from the elongate member via unlooping the elongate member from the bight.

Example 76. The system according to example 75, wherein, in the delivery state, both a first end and a second end of the elongate member are coupled to the extracorporeal control portion.

Example 77. The system according to any one of examples 75-76, wherein: (A) the implant further comprises a first component and a second component, the tether tethering the first component to the second component, and (B) the delivery tool is configured to: (i) anchor the first component at a first site within the heart, and (ii) subsequently, advance the second component away from the first site toward a second site within the heart while (a) the first component remains anchored at the first site, and (b) the bight of the tether remains confined within the channel by the elongate member being looped onto the bight.

Example 78. The system according to example 77, wherein the second component comprises a winch, operatively coupled to the tether.

Example 79. The system according to example 77, wherein: (i) the first site is at a leaflet of a valve of the heart, (ii) the first component is a leaflet anchor adapted to be anchored to the leaflet, (iii) the second site is in a ventricle downstream of the valve, (iv) the second component is a ventricular anchor adapted to be anchored to tissue of the ventricle, and (v) the delivery tool is configured to transluminally advance the ventricular anchor towards the second site in the ventricle while the first component remains anchored to the leaflet.

Example 80. The system according to any one of examples 75-79, wherein the delivery tool comprises a tube, the channel is fixed with respect to the tube, and the shaft extends through the tube, and is configured to transluminally position the tether within the heart by the shaft sliding distally through the tube in a manner that pulls the tether distally out of the distal opening of the channel.

Example 81. The system according to example 80, wherein the tube is shaped to define the channel.

Example 82. The system according to example 80, wherein the extracorporeal control portion is coupled to a proximal part of the tube and is configured to slide the shaft distally through the tube.

Example 83. The system according to any one of examples 75-82, wherein the elongate member is reversibly coupled to a deformable member that deforms in response to pulling of the tether toward the distal opening.

Example 84. The system according to example 83, wherein the deformable member is at the extracorporeal control portion.

Example 85. The system according to example 83, wherein the deformable member is elastically deformable in a manner that configures the deformable member to draw the bight back into the channel responsively to relaxing of the pulling of the tether.

Example 86. The system according to example 83, wherein the deformable member is plastically deformable responsively to the pulling of the tether.

Example 87. The system according to example 83, wherein the deformable member comprises a spring that deforms in response to pulling of the tether toward the distal opening.

Example 88. The system according to example 87, wherein the deformable member comprises a torsion spring.

Example 89. The system according to example 83, wherein the extracorporeal control portion comprises a bobbin, operatively coupled to the elongate member in a manner that configures the bobbin to unloop the elongate member from the bight by winding the elongate member around the bobbin.

Example 90. The system according to example 89, wherein the deformable member comprises a torsion spring.

Example 91. The system according to example 90, wherein the torsion spring is disposed within the bobbin.

Example 92. The system according to example 90, wherein the torsion spring is disposed around the bobbin.

Example 93. The system according to any one of examples 75-92, wherein the part of the elongate member is coupled to the extracorporeal control portion in a manner that exposes the part of the elongate member, such that the part of the elongate member is decouplable from the extracorporeal control portion by cutting the part of the elongate member.

Example 94. The system according to example 93, wherein: (i) the part of the elongate member is decouplable from the extracorporeal control portion by cutting the part of the elongate member thereby producing a cut end of the elongate member, and/or (ii) the part of the elongate member is decouplable from the extracorporeal control portion to facilitate the release of the bight from the elongate member via drawing the cut end distally through the channel such that the elongate member unloops from the bight.

Example 95. A method, the method comprising: (A) using a delivery tool that defines a channel at a distal portion thereof, advancing a tether to a heart while: (i) a bight of the tether is retained within the channel by an elongate member looped onto the bight within the channel, and (ii) at least part of the elongate member is coupled to an extracorporeal control portion of the delivery tool; (B) while the distal portion of the delivery tool is disposed within the heart, progressively pulling the tether out of an opening of the channel such that the elongate member moves towards the opening of the channel; and (C) subsequently, releasing the tether from the channel by: (i) decoupling at least a part of the elongate member from the extracorporeal control portion, and (ii) drawing the part of the elongate member distally through the channel such that the elongate member unloops from the bight.

Example 96. The method according to example 95, wherein the extracorporeal control portion comprises a bobbin, operatively coupled to the elongate member, and wherein drawing the part of the elongate member distally through the channel comprises winding the elongate member around the bobbin such that the part of the elongate member is drawn distally into the channel.

Example 97. The method according to example 95, wherein decoupling the part of the elongate member from the extracorporeal control portion comprises cutting the part of the elongate member.

Example 98. The method according to any one of examples 95-97, wherein advancing the tether to the heart comprises advancing the tether to the heart while both a first end and a second end of the elongate member are coupled to the extracorporeal control portion.

Example 99. The method according to example 98, wherein the extracorporeal control portion comprises a bobbin, operatively coupled to the first end of the elongate member, and wherein drawing the part of the elongate member distally through the channel comprises drawing the second end of the elongate member distally through the channel by winding the elongate member around the bobbin.

Example 100. The method according to example 98, wherein the part of the elongate member is the second end of the elongate member, and wherein decoupling the part of the elongate member from the extracorporeal control portion comprises cutting the second end of the elongate member.

Example 101. The method according to any one of examples 95-100, wherein the elongate member is a component of a retaining member that further comprises a deformable member coupled to the elongate member, and/or wherein progressively pulling the tether out of the opening of the channel comprises progressively pulling the tether out of the opening of the channel such that the deformable member deforms as the elongate member moves towards the opening.

Example 102. The method according to example 101, wherein the deformable member is elastically deformable in a manner that configures the deformable member to draw the bight back into the channel responsively to relaxing of the pulling of the tether.

Example 103. The method according to example 101, wherein the deformable member comprises a spring that deforms in response to pulling of the tether toward the distal opening, and/or wherein progressively pulling the tether out of the opening of the channel comprises progressively pulling the tether out of the opening of the channel such that the spring deforms as the elongate member moves towards the opening.

Example 104. The method according to any one of examples 95-103, wherein the tether is a component of an implant, the implant further includes an anchor coupled to the tether, and/or the method further comprises using the delivery tool to anchor the anchor to tissue of the heart.

Example 105. The method according to example 104, wherein anchoring the anchor to tissue of the heart comprises anchoring the anchor to tissue of the heart subsequently to releasing the tether from the channel.

Example 106. The method according to example 104, wherein anchoring the anchor to tissue of the heart comprises anchoring the anchor to tissue of the heart prior to releasing the tether from the channel.

Example 107. The method according to example 104, wherein the implant further includes a winch, and wherein anchoring the anchor comprises anchoring the winch to the tissue by anchoring the anchor to the tissue.

Example 108. The method according to example 107, further comprising, subsequently to anchoring the winch to the tissue, adjusting an amount of the tether that is let out from the winch by actuating the winch.

Example 109. The method according to example 107, further comprising, subsequently to anchoring the winch to the tissue, adjusting tension on the tether by actuating the winch.

Example 110. The method according to example 104, wherein the anchor is a second anchor, the implant further including a first anchor coupled to the tether, and/or anchoring the anchor comprises anchoring the second anchor at a second site within the heart, such that the tether extends from the second anchor, anchored at the second site, to the first anchor.

Example 111. The method according to example 110, further comprising anchoring the first anchor at a first site within the heart, prior to anchoring the second anchor at the second site.

Example 112. The method according to example 111, wherein progressively pulling the tether out of the opening of the channel comprises progressively pulling the tether out of the opening of the channel by advancing the second anchor away from the first site toward the second site, subsequently to anchoring the first anchor.

The treatment techniques, methods, operations, steps, etc. described or suggested herein or in the references incorporated herein can be performed on a living subject (e.g., human, other animal, etc.) or on a non-living simulation, such as a cadaver, cadaver heart, simulator, imaginary person, etc. When performed on a simulation, the body parts, e.g., heart, tissue, valve, etc., can optionally be referred to as “simulated” (e.g., simulated heart, simulated tissue, simulated valve, etc.) and can comprise, for example, computerized and/or physical representations of body parts, tissue, etc.

Any of the above systems, assemblies, devices, apparatuses, components, etc. can be sterilized (e.g., with heat, radiation, ethylene oxide, hydrogen peroxide, etc.) to ensure they are safe for use with patients, and the above methods can comprise (or additional methods comprise or consist of) sterilization of one or more systems, devices, apparatuses, components, etc. herein (e.g., with heat, radiation, ethylene oxide, hydrogen peroxide, etc.).

The present invention is not limited to the examples that have been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

What is claimed is:

1. A system comprising:
an implant, comprising:
a winch, comprising a spool; and
a tether, coupled to the spool; and
a delivery tool, configured to transluminally deliver the implant to a heart, and comprising:
a shaft, coupled to the winch, and configured to transluminally position the winch within the heart,
a handle, comprising a winch controller, and coupled to a proximal part of the shaft such that distal movement of the handle moves the winch distally into the heart, the proximal part of the shaft defining an axis of the delivery tool, and
a depth-stopper, extending axially from the handle,
wherein the winch controller is operatively coupled to the winch and to the depth-stopper such that operation of the winch controller in a manner that adjusts an amount of winding of the tether around the spool, also adjusts axial extension of the depth-stopper from the handle.

2. The system according to claim 1, wherein the depth-stopper comprises a rod.

3. The system according to claim 1, wherein the depth-stopper extends parallel to the axis.

4. The system according to claim 1, wherein the shaft is configured to transluminally position the winch within the heart while extending, from the handle, through vasculature of a subject, to the winch within the heart.

5. The system according to claim 1, wherein the winch controller comprises a control knob, and is operatively coupled to the winch and to the depth-stopper such that rotation of the control knob in a manner that adjusts an amount of winding of the tether around the spool, also adjusts axial extension of the depth-stopper from the handle.

6. The system according to claim 1, wherein the winch controller is operatively coupled to the winch and to the depth-stopper via a rotary-to-linear mechanism.

7. The system according to claim 1, wherein the implant comprises an anchor, coupled to the winch, and configured to anchor the winch to tissue at a site in the heart.

8. The system according to claim 7, wherein:
the anchor is a second anchor,
the site is a second site, and
the implant further comprises a first anchor, coupled to a first end portion of the tether, and configured to anchor the first end portion of the tether to tissue at a first site within the heart, and
the shaft is configured to transluminally advance the winch and the second anchor towards the second site while the first anchor remains anchored at the first site.

9. The system according to claim 1, wherein the delivery tool has a state in which the depth-stopper extends distally from the handle.

10. The system according to claim 9, wherein the winch controller is operatively coupled to the winch and to the depth-stopper such that operation of the winch controller in a manner that decreases the amount of winding of the tether around the spool, also moves the depth-stopper proximally with respect to the handle.

11. The system according to claim 10, wherein the winch controller is operatively coupled to the winch and to the depth-stopper such that operation of the winch controller in a manner that increases the amount of winding of the tether around the spool, also moves the depth-stopper distally with respect to the handle.

12. The system according to claim 9, wherein the winch controller is operatively coupled to the winch and to the depth-stopper such that operation of the winch controller in a manner that decreases the amount of winding of the tether around the spool, also moves the depth-stopper distally with respect to the handle.

13. The system according to claim 12, wherein the winch controller is operatively coupled to the winch and to the depth-stopper such that operation of the winch controller in a manner that increases the amount of winding of the tether around the spool, also moves the depth-stopper proximally with respect to the handle.

14. The system according to claim 9, wherein the state is a first state, and wherein the delivery tool has a second state in which the depth-stopper extends proximally from the handle.

15. The system according to claim 1, further comprising a mount in which the handle is mountable, and which, while the handle is mounted in the mount, is configured to obstruct axial movement of the depth-stopper along an axial path by being disposed in the axial path.

16. The system according to claim 15, wherein the winch controller is operatively coupled to the winch and to the depth-stopper such that:
operation of the winch controller in a manner that decreases an amount of winding of the tether around the spool, also moves the depth-stopper proximally with respect to the mount, and
operation of the winch controller in a manner that increases an amount of winding of the tether around the spool, also moves the depth-stopper distally with respect to the mount.

17. The system according to claim 15, wherein the winch controller is operatively coupled to the winch and to the depth-stopper such that:
operation of the winch controller in a manner that increases an amount of winding of the tether around the spool, also moves the depth-stopper proximally with respect to the mount, and
operation of the winch controller in a manner that decreases an amount of winding of the tether around the spool, also moves the depth-stopper distally with respect to the mount.

18. The system according to claim 15, wherein the winch controller is operatively coupled to the winch and to the depth-stopper such that operation of the winch controller in the manner that adjusts the amount of winding of the tether around the spool, also adjusts an axial position of the depth-stopper with respect to the mount.

19. The system according to claim 18, wherein the mount comprises:
a distal stop that, while the handle is mounted in the mount, is positioned to obstruct movement of the depth-stopper distally by being disposed in the axial path; and
a proximal stop that, while the handle is mounted in the mount, is positioned to obstruct movement of the depth-stopper proximally by being disposed in the axial path.

20. A system for controlling slack in a tether during implantation of the tether in a heart of a subject, the system comprising:
the tether, the tether having a first end portion, a second end portion, and a bight therebetween, and
a delivery tool, configured to secure the first end portion of the tether and the second end portion of the tether within the heart, and comprising:
a channel having an inner wall and a distal opening, and
a retaining member, constrained by the inner wall to define a hook within the channel, wherein:
the bight of the tether is confined within the channel by being hooked onto the hook;
the retaining member is deformable responsively to pulling of the tether out of the distal opening in a manner that moves the hook toward the distal opening, and
the retaining member is configured such that, upon the hook exiting the channel via the distal opening, the hook responsively opens and unhooks from the bight.
